(19) United States
(12) Patent Application Publication
(10) Pub. No.: US 2007/0122414 A1
(43) Pub. Date: May 31, 2007

(54) Surface marker-directed cancer therapeutics

(75) Inventors: Elias Georges, Laval (CA); Anne-Marie Bonneau, Laval (CA)

Correspondence Address: WILMER CUTLER PICKERING HALE AND DORR LLP, 60 STATE STREET, BOSTON, MA, 02109, US

(73) Assignee: Aurelium BioPharma Inc., Montreal (CA)

(21) Appl. No.: 11/595,299

(22) Filed: Nov. 9, 2006

Related U.S. Application Data
(60) Provisional application No. 60/735,543, filed on Nov. 10, 2005.
(60) Provisional application No. 60/796,638, filed on May 1, 2006.

(51) Int. Cl. A61K 39/395; A61K 38/16; A61K 31/7072; A61K 31/704; A61K 48/00; A61K 31/4745; A61K 31/282

(52) U.S. Cl. 424/155.1; 424/450; 424/178.1; 514/8; 514/34; 514/49; 514/410; 514/109; 514/263.31; 514/449; 514/575; 514/283; 514/492

(57) ABSTRACT

Disclosed are methods for treating and/or preventing neoplasms in a patient by contacting the neoplasm with therapeutic agents capable of binding, hybridizing, or interacting with proteins on the cell surface of neoplastic cells. In addition, therapeutic compositions are disclosed for the treatment and/or prevention of a neoplasm in a patient in need thereof.

This Application claims the benefit of priority to U.S. Provisional Application No. 60/735,543, filed Nov. 10, 2005 and to U.S. Provisional Application No. 60/796,638, filed May 1, 2006, the specifications of which are incorporated by reference in their entirety.

### FIELD OF THE INVENTION

The present invention relates generally to the field of medicine. More specifically, the invention pertains to methods and formulations for the treatment of cancer in a subject.

### BACKGROUND OF THE INVENTION

Cancer is one of the deadliest illnesses in the United States, accounting for nearly 600,000 deaths annually. This disorder is in fact a diverse group of diseases, which can originate in almost any tissue of the body. In addition, cancers may be generated by multiple mechanisms including pathogenic infections, mutations, and environmental insults (see, e.g., Pratt et al. (2005) Hum. Pathol. 36(8): 861-70). The variety of cancer types and mechanisms of tumorigenesis add to the difficulty associated with treating a tumor, increasing the risk posed by the cancer to the patient's life and wellbeing.

Diseases such as cancer are often treated with drugs (e.g., chemotherapeutics and antibiotics). In order to kill the cancer or diseased cells, the drug(s) must enter the cells and reach an effective dose so as to interfere with essential biochemical pathways. However, some cells evade being killed by the drug by developing resistance to it (termed “drug resistance”). Moreover, in some cases, cancer cells (also called tumor cells or neoplastic cells) develop resistance to a broad spectrum of drugs, including drugs that were not originally used for treatment. This phenomenon is termed “chemotherapeutic drug resistance.” There are different types of chemotherapeutic drug resistance, each associated with a different biological mechanism, and there are specific biological “markers” for different types of chemotherapeutic drug resistance.

In addition to the emergence of chemotherapeutic drug resistance, cancer cells have inherent differences in their sensitivities to particular chemotherapeutic drugs. These characteristics depend on several factors including the genetic background of the cell. In particular, certain genes expressed by a cancer cell are significantly different from those expressed by a normal cell of the tissue in which the cancer cell is derived.

Many genes showing differential expression in cancer cells are expressed on the cell surface. In some cases, cancer cells express cell surface proteins that are not found on the surface of normal cells. For instance, small cell carcinoma of the lung expresses at least twelve proteins on the cell surface that are not found on the surface of normal cells of the lung (Baylin et al. (1982) Proc. Nat. Acad. Sci. USA 79(15): 4650-54). Some of these cell surface proteins provide cancer cells with resistance to chemotherapeutic drugs, as in the case of Pgp-1 (see, e.g., Volm et al., (1993) Cancer 71: 3981-3987; Bradley and Ling, Cancer Metastasis Rev. (1994) 13: 223-233). In other instances, these proteins endow the cancer cells with the ability to perform certain functions such as invasion of surrounding tissues (see, e.g., Nomura and Katunuma (2005) J. Med. Invest. 52(1-2): 1-9) of increase in cell adhesion (Nieminen et al. Nature. cell Biology (2006):8 156-162).

There remains a need in both humans and animals for treating, preventing, and reversing the development of neoplastic cells. In addition, the ability to identify and to make use of reagents that identify multiple drug resistant cells has clinical potential for improvements in the treatment and monitoring of cancer. By facilitating the treatment efficacy of chemotherapeutic drugs, there is a potential for significant improvements in the quality of life and survival of patients diagnosed with cancer.

### SUMMARY OF THE INVENTION

The present invention is based, in part, upon the discovery that cancer cells express certain proteins or combinations of proteins on their cell surface that normal cells do not express. This discovery has been exploited to develop the present invention that, in part, provides therapeutic methods that use therapeutic agents specific for particular cell surface proteins to treat, prevent, or reverse the development of neoplastic cells in a subject. Moreover, the therapeutic methods can increase the sensitivity of the neoplastic cells to chemotherapeutic drugs. The invention also provides therapeutic methods for treating cells that have developed chemotherapeutic drug resistance.

In one aspect, the invention provides a method of treating and/or preventing a neoplasm in a patient. The method comprises administering an effective amount of at least one therapeutic agent to a patient in which the therapeutic agent is capable of binding to at least one protein marker.

In another aspect, the invention provides a method of treating and/or preventing a neoplasm in a patient in need thereof. The method comprises administering an effective amount of one or more therapeutic agents, each of which binds specifically to a protein target from the group consisting of nucleophosmin, HSC70, BIP, Grp75, PDI ER60 precursor, HSP60, TCP-1ε, ERp29, HSP27, vimentin, α-internexin, cytokeratin 8, β-actin, γ-actin, β-tubulin, nm23-H1, valosin containing protein, tumor protein D52-like 2, ASF-2, hnRNPK, hnRNPC, 24.1D5 antigen, hnRNPA/B, Eukaryotic Elongation Factor 1δ Isoform 2, AU-rich element RNA binding protein, Rad 23 homologue B, annexin I, prohibitin, ubiquilin 1, and thioredoxin peroxidase 4. The protein target is expressed on the surface of the neoplasm. In certain embodiments, the therapeutic agents are from the group consisting of ligands, nucleic acids, synthetic small molecules, peptidomimetic compounds, inhibitors, peptides, proteins, antibodies, antigen-binding fragments of antibodies, and combinations thereof. In other embodiments, two or more therapeutic agents are administered simultaneously. In still other embodiments, the therapeutic agent bound to the protein target on the neoplasm is internalized by the neoplastic cell.

In certain embodiments, the one or more therapeutic agents are incorporated into a liposome prior to administration. In other embodiments, the liposome comprises a neoplastic cell-targeting agent on its surface. In particular embodiments, the neoplastic cell-targeting agent comprises an antibody, or antigen-binding fragment thereof, specific for at least one cell marker selected from the group consisting of multidrug resistance protein 1, BRCP, p53, vimentin, α-enolase, nucleophosmin, and HSC70.

In certain embodiments, the therapeutic agent(s) is administered to the patient by injection at the site of the neoplasm or intravenously. In other embodiments, the therapeutic agent(s) is administered to the patient by surgical introduction at the site of the neoplasm. In still other embodiments, the therapeutic agent(s) is administered to the patient by inhalation of an aerosol or vapor or via a transdermal patch.

In some embodiments, two or more therapeutic agents that bind to two or more of the protein targets are administered to a patient in need thereof. In other embodiments, the neoplasm is selected from the group consisting of breast adenocarcinoma, breast carcinoma, ovarian carcinoma, ovarian adenocarcinoma, lung small cell carcinoma, lung carcinoma, and leukemia. In still other embodiments, the neoplasm comprises breast adenocarcinoma. In yet other embodiments, the neoplasm comprises ovarian adenocarcinoma.

In further embodiments, three or more therapeutic agents that bind to three or more of the protein targets are administered to a patient in need thereof. In other embodiments, four or more therapeutic agents that bind to four or more of the protein targets are administered to a patient in need thereof. In still other embodiments, five or more therapeutic agents that bind to five or more of the protein targets are administered to a patient in need thereof. In particular embodiments, six or more therapeutic agents that bind to six or more of the protein targets are administered to a patient in need thereof. In more particular embodiments, seven or more therapeutic agents that bind to seven or more of the protein targets are administered to a patient in need thereof. In still more particular embodiments, eight or more therapeutic agents that bind to eight or more of the protein targets are administered to a patient in need thereof.

In further embodiments, nine or more therapeutic agents that bind to nine or more of the protein targets are administered to a patient in need thereof. In other embodiments, ten or more therapeutic agents that bind to ten or more of the protein targets are administered to a patient in need thereof. In still other embodiments, 11 or more therapeutic agents that bind to 11 or more of the protein targets are administered to a patient in need thereof. In yet other embodiments, 12 or more therapeutic agents that bind to 12 or more of the protein targets are administered to a patient in need thereof. In further embodiments, 13 or more therapeutic agents that bind to 13 or more of the protein targets are administered to a patient in need thereof. In still further embodiments, 14 or more therapeutic agents that bind to 14 or more of the protein targets are administered to a patient in need thereof.

In additional embodiments, 15 or more therapeutic agents that bind to 15 or more of the protein targets are administered to a patient in need thereof. In other embodiments, 16 or more therapeutic agents that bind to 16 or more of the protein targets are administered to a patient in need thereof. In yet other embodiments, 17 or more therapeutic agents that bind to 17 or more of the protein targets are administered to a patient in need thereof. In more embodiments, 18 or more therapeutic agents that bind to 18 or more of the protein targets are administered to a patient in need thereof. In still more embodiments, 19 or more therapeutic agents that bind to 19 or more of the protein targets are administered to a patient in need thereof.

In still further embodiments, 20 or more therapeutic agents that bind to 20 or more of the protein targets are administered to a patient in need thereof. In other embodiments, 21 or more therapeutic agents that bind to 21 or more of the protein targets are administered to a patient in need thereof. In yet other embodiments, 22 or more therapeutic agents that bind to 22 or more of the protein targets are administered to a patient in need thereof.

In other embodiments, 23 or more therapeutic agents that bind to 23 or more of the protein targets are administered to a patient in need thereof. In still other embodiments, 24 or more therapeutic agents that bind to 24 or more of the protein targets are administered to a patient in need thereof. In still other embodiments, 25 or more therapeutic agents that bind to 25 or more of the protein targets are administered to a patient in need thereof. In more embodiments, 26 or more therapeutic agents that bind to 26 or more of the protein targets are administered to a patient in need thereof. In still more embodiments, 27 or more therapeutic agents that bind to 27 or more of the protein targets are administered to a patient in need thereof. In yet more embodiments, 28 or more therapeutic agents that bind to 28 or more of the protein targets are administered to a patient in need thereof. In still further embodiments, 29 or more therapeutic agents that bind to 29 or more of the protein targets are administered to a patient in need thereof. In particular embodiments, all therapeutic agents directed against the protein targets are administered to a patient in need thereof.

In certain embodiments, one or more therapeutic agents are administered simultaneously or sequentially with a chemotherapeutic drug. In other embodiments, the chemotherapeutic drug is selected from the group consisting of Actinomycin, Adriamycin, Altretamine, Asparaginase, Bleomycin, Busulfan, Capecitabine, Carboplatin, Carmustine, Chlorambucil, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastine, Vincristine, Vinorelbine, and combinations thereof.

In yet another aspect, the invention provides a cell surface-targeting agent for treating or preventing a neoplasm. The cell surface-targeting agent comprises a cell surface protein-binding component and a therapeutic component. In such an aspect, the cell surface protein-binding component targets the therapeutic component to the neoplasm by binding to the cell surface proteins selected from the group consisting of nucleophosmin, HSC70, BIP, Grp75, PDI ER60 precursor, HSP60, TCP-1ε, ERp29, HSP27, vimentin, α-internexin, cytokeratin 8, β-actin, γ-actin, β-tubulin, nm23-H1, valosin containing protein, tumor protein D52-like 2, ASF-2, hnRNPK, hnRNPC, 24.1D5 antigen, hnRNPA/B, Eukaryotic Elongation Factor 1δ Isoform 2, AU-rich element RNA binding protein, Rad 23 homologue B, annexin I, prohibitin, ubiquilin 1, and thioredoxin peroxidase 4.

In some embodiments, the cell surface-protein targeting component is selected from the group consisting of ligands, nucleic acids, synthetic small molecules, peptidomimetic compounds, inhibitors, peptides, proteins, and antibodies or antigen-binding fragments thereof. In certain embodiments, the cell surface-targeting component comprises an antibody, or antigen-binding fragment thereof, directed against at least one cell surface protein. In other embodiments, the therapeutic component is selected from the group consisting of Actinomycin, Adriamycin, Altretamine, Asparaginase, Bleomycin, Busulfan, Capecitabine, Carboplatin, Carmustine, Chlorambucil, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastine, Vincristine, Vinorelbine, and combinations thereof.

In some embodiments, the therapeutic component is in a liposome formulation. In other embodiments, the therapeutic component is a radioisotope. In certain embodiments, the radioisotope is selected from the group consisting of ⁹⁰Y, ¹²⁵I, ¹³¹I, ²¹¹At, and ²¹³Bi. In further embodiments, the therapeutic component is a toxin capable of killing or inducing the killing of the targeted neoplastic cell.

In another aspect, the invention provides a pharmaceutical formulation for treating a neoplasm. The formulation comprises a plurality of therapeutic agents directed against a plurality of cell surface protein targets selected from the group comprising nucleophosmin, HSC70, BIP, Grp75, PDI ER60 precursor, HSP60, TCP-1ε, ERp29, HSP27, vimentin, α-internexin, cytokeratin 8, β-actin, γ-actin, β-tubulin, nm23-H1, valosin containing protein, tumor protein D52-like 2, ASF-2, hnRNPK, hnRNPC, 24.1D5 antigen, hnRNPA/B, Eukaryotic Elongation Factor 1δ Isoform 2, AU-rich element RNA binding protein, Rad 23 homologue B, annexin I, prohibitin, ubiquilin 1, and thioredoxin peroxidase 4. The pharmaceutical formulation further comprises a chemotherapeutic drug and a pharmaceutically acceptable carrier.

In some embodiments, at least 3 therapeutic agents are selected, and each therapeutic agent binds to at least one surface protein target. In certain embodiments, the therapeutic agents are selected from the group consisting of ligands, nucleic acids, synthetic small molecules, peptidomimetic compounds, inhibitors, peptides, proteins, antibodies, antigen-binding fragments of antibodies, and combinations thereof. In other embodiments, the therapeutic agents are incorporated into a liposome. In still other embodiments, the liposome comprises a neoplastic cell-targeting component on its surface. In particular embodiments, the neoplastic cell-targeting component is an antibody, or antigen-binding fragment thereof, that binds to a neoplastic cell marker selected from the group consisting of multidrug resistance protein 1, BRCP, p53, vimentin, α-enolase, nucleophosmin, and HSC70. In more particular embodiments, the chemotherapeutic drug is selected from the group consisting of Actinomycin, Adriamycin, Altretamine, Asparaginase, Bleomycin, Busulfan, Capecitabine, Carboplatin, Carmustine, Chlorambucil, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastine, Vincristine, Vinorelbine, and combinations thereof.

In yet another aspect, the invention provides a method of treating and/or preventing a breast neoplasm in a patient. The method comprises administering an effective amount of at least six therapeutic agents directed against protein targets from the group consisting of nucleophosmin, HSC70, BIP, Grp75, PDI ER60 precursor, HSP60, TCP-1ε, ERp29, HSP27, vimentin, α-internexin, cytokeratin 8, β-actin, γ-actin, β-tubulin, nm23-H1, valosin containing protein, tumor protein D52-like 2, ASF-2, hnRNPK, hnRNPC, 24.1D5 antigen, hnRNPA/B, Eukaryotic Elongation Factor 1δ Isoform 2, AU-rich element RNA binding protein, Rad 23 homologue B, annexin I, prohibitin, ubiquilin 1, and thioredoxin peroxidase 4, wherein the therapeutic agents increase the sensitivity of the neoplasm to a chemotherapeutic treatment.

In yet another aspect, the invention provides a method of treating and/or preventing an ovarian neoplasm in a patient. The method comprises administering an effective amount of at least six therapeutic agents directed against protein targets from the group consisting of nucleophosmin, HSC70, BIP, Grp75, PDI ER60 precursor, HSP60, TCP-1ε, ERp29, HSP27, vimentin, α-internexin, cytokeratin 8, β-actin, γ-actin, β-tubulin, nm23-H1, valosin containing protein, tumor protein D52-like 2, ASF-2, hnRNPK, hnRNPC, 24.1D5 antigen, hnRNPA/B, Eukaryotic Elongation Factor 1δ Isoform 2, AU-rich element RNA binding protein, Rad 23 homologue B, annexin I, prohibitin, ubiquilin 1, and thioredoxin peroxidase 4, wherein the therapeutic agents increase the sensitivity of the neoplasm to a chemotherapeutic treatment.

In yet another aspect, the invention provides a method of treating and/or preventing leukemia in a patient. The method comprises administering an effective amount of at least six therapeutic agents directed against protein targets from the group consisting of nucleophosmin, HSC70, BIP, Grp75, PDI ER60 precursor, HSP60, TCP-1ε, ERp29, HSP27, vimentin, α-internexin, cytokeratin 8, β-actin, γ-actin, β-tubulin, nm23-H1, valosin containing protein, tumor protein D52-like 2, ASF-2, hnRNPK, hnRNPC, 24.1D5 antigen, hnRNPA/B, Eukaryotic Elongation Factor 1δ Isoform 2, AU-rich element RNA binding protein, Rad 23 homologue B, annexin I, prohibitin, ubiquilin 1, and thioredoxin peroxidase 4, wherein the therapeutic agents increase the sensitivity of the neoplasm to a chemotherapeutic treatment.

In yet another aspect, the invention provides a method of treating and/or preventing a lung neoplasm in a patient. The method comprises administering an effective amount of at least six therapeutic agents directed against protein targets from the group consisting of nucleophosmin, HSC70, BIP, Grp75, PDI ER60 precursor, HSP60, TCP-1ε, ERp29, HSP27, vimentin, α-internexin, cytokeratin 8, β-actin, γ-actin, β-tubulin, nm23-H1, valosin containing protein, tumor protein D52-like 2, ASF-2, hnRNPK, hnRNPC, 24.1D5 antigen, hnRNPA/B, Eukaryotic Elongation Factor 1δ Isoform 2, AU-rich element RNA binding protein, Rad 23 homologue B, annexin I, prohibitin, ubiquilin 1, and thioredoxin peroxidase 4, wherein the therapeutic agents increase the sensitivity of the neoplasm to a chemotherapeutic treatment.

### BRIEF DESCRIPTION OF THE FIGURES

FIG. 1 is a graphic representation of the results of treatments using either vimentin alone, or in combination with taxol, on the size of SKOV3 tumor size in nude mice.

FIG. 2 is a graphic representation of the results of treatments using either vimentin alone, or in combination with taxol, on the size of SKOV3 tumor size in nude mice.

### DETAILED DESCRIPTION OF THE INVENTION

The patent and scientific literature referred to herein establishes knowledge that is available to those of skill in the art. The issued US patents, allowed applications, published foreign applications, and references, including GenBank database sequences, that are cited herein are hereby incorporated by reference to the same extent as if each was specifically and individually indicated to be incorporated by reference.

1.1 General

Aspects of the present invention provide methods and reagents for preventing and treating the development of cancer in a patient. In some aspects, the neoplasm is made more sensitive to the chemotherapeutic treatment by decreasing the level of expression of cell surface proteins on the neoplastic cells. Other aspects of the invention provide methods and reagents to treat and/or prevent the development of cancer in a patient. Additionally, the invention allows for the improved clinical efficacy of chemotherapeutic drugs against tumors.

Accordingly, the invention provides, in part, methods for treating and/or preventing a neoplastic cell in a patient. In particular methods, the invention allows for treating neoplastic cells using therapeutic agents against cell surface proteins that are typically associated with neoplasms. Therapeutic agents can be administered in amounts necessary to either kill the neoplastic cell or increase the effectiveness of chemotherapeutic treatments.

As used herein, the term “therapeutic agent” means a compound that is capable of binding, hybridizing, associating, or interacting with a target compound. Therapeutic agents can specifically associate with target proteins, with mRNA encoding the target protein, or with other components of the cellular machinery that prevent cell surface expression of the target proteins. Examples of therapeutic agents include, but are not limited to, ligands, small molecules, organic molecules, peptidomimetic compounds, inhibitors, nucleic acids, aptamers, proteins, peptides, and antibodies or antigen-binding fragments thereof.

As used herein, by the term “target protein” is meant a protein, polypeptide, or peptide fragment thereof, that is the target of at least one therapeutic agent.

As used herein, the term “binding” means interacting with a molecule or group of molecules to produce a relatively stable association between molecules. Examples of molecular binding include hydrogen bonding, London forces, van der Waals interactions, covalent bonding, and ionic bonding. Single or multiple binding interactions are useful to produce with molecules.

As used herein, the term “cell surface protein” means a protein that is associated with the cell membrane by either hydrophobic interactions, covalent attachment, London forces, hydrogen bonding, or is integrally associated with the lipid bilayer. Specific examples of cell surface proteins that are included within the scope of the present invention are listed in Table 1.

TABLE 1
Cell Surface Protein Targets of Therapeutic Agents

| | | | | |
|---|---|---|---|---|
| ASF-2 | valosin contain- | HSP27 | annexin I | prohibitin |
| | ing protein | | | |
| Grp75 | 24.1D5 antigen | hnRNPC | D52-like 2 | ubiquilin 1 |
| HnRNPA/B | nm23-H1 | hnRNPK | BIP | ERp29 |
| TCP-1 ε | Thioredoxin | nucleophosmin | EEF1 δ | HSC70 |
| | peroxidase 4 | | Isoform 2 | |
| α-internexin | PDI ER60 | β-actin | γ-actin | β-tubulin |
| | precursor | | | |
| cytokeratin 8 | AU-rich | Rad 23 | HSP60 | vimentin |
| | element RNA- | hom B | | |
| | binding prot. | | | |

As used herein, a “neoplastic cell” is a cell that shows aberrant cell growth, such as increased, uncontrolled cell growth. A neoplastic cell can be a hyperplastic cell, a cell from a cell line that shows a lack of contact inhibition when grown in vitro, a tumor cell when grown in vivo, or a cancer cell that is capable of metastasis in vivo. Alternatively, a neoplastic cell can be termed a “cancer cell.” Non-limiting examples of cancer cells include melanoma, breast cancer, ovarian cancer, prostate cancer, sarcoma, leukemic retinoblastoma, hepatoma, myeloma, glioma, mesothelioma, carcinoma, leukemia, lymphoma, Hodgkin lymphoma, Non-Hodgkin lymphoma, promyelocytic leukemia, lymphoblastoma, and thymoma, and lymphoma cells, melanoma cells, sarcoma cells, leukemia cells, retinoblastoma cells, hepatoma cells, myeloma cells, glioma cells, mesothelioma cells, and carcinoma cells.

Cancer cells can be obtained from non-limiting tissues such as breast, lung, bone, blood, skin, brain, gastrointestinal, lymphatic, hepatic, muscle, ovary, uterine, and kidney. Cancer cells can be obtained from tissues other than the tissue from which the cancer cell originally developed, as in the case of metastasized cancer cells. Moreover, cancer cells can be obtained from mammals including, but not limited to, human, non-human primates such as chimpanzee, mouse, rat, guinea pig, chinchilla, rabbit, pig, and sheep.

As used herein, the term “normal cell” means a cell that exhibits the characteristics expected for a non-cancerous cell of its particular tissue type, age, developmental stage, and organism. A normal cell generally exhibits growth characteristics that are not aberrant when compared to the cells of its particular tissue type, age, developmental stage, and organism. Normal cells do not tend to harm the functionality of the tissue from which they are isolated. In addition, normal cells do not show uncontrolled growth within the organism. “Uncontrolled growth” is defined as proliferation that is outside the normal range exhibited by cells of a particular tissue type, age, developmental stage, and organism.

As used herein, “chemotherapeutic drug” means a pharmaceutical compound that kills a damaged cell such as a cancer cell. Cell death can be induced by the chemotherapeutic drug through a variety of means including, but not limited to, apoptosis, osmolysis, electrolyte efflux, electrolyte influx, cell membrane permeablization, and DNA fragmentation. Exemplary non-limiting chemotherapeutic drugs are adriamycin, cisplatinum, taxol, melphalan, daunorubicin, dactinomycin, bleomycin, fluorouracil, teniposide, vinblastin, vincristine, methotrexate, mitomycin, docetaxel, chlorambucil, carmustine, mitoxantrone, and paclitaxel.

As used herein, the term “chemotherapeutic drug-resistance” encompasses the development of resistance to a particular chemotherapeutic drug, class of chemotherapeutic drugs or multiple chemotherapeutic drugs by a cancer cell. Resistance can occur before or after treatment with a chemotherapy regime. Without being limited to any one theory, the mechanism of development of chemotherapeutic drug resistance can occur by any means, such as by pathogenic means such as through infections, particularly viral infection. Alternatively, chemotherapeutic drug resistance can be conferred by a mutation or mutations in one or several genes located either chromosomally or extrachromosomally. In addition, chemotherapeutic drug resistance can be conferred by selection of a certain phenotype by exposure to the chemotherapeutic drug or class of chemotherapeutic drugs, and then subsequent survival of the cell to the particular treatment. The above-mentioned mechanisms of chemotherapeutic drug resistance are known in the art. The terms, “chemotherapeutic drug-resistant” and “chemotherapeutic drug resistance,” are used to describe a neoplastic cell or a damaged cell that is resistant or relatively unaffected y such drugs, either the classical mechanism (i.e., involving P-glycoprotein or another MDR protein) or an atypical mechanism (non-classical mechanism) that does not involve P-glycoprotein (e.g., an atypical mechanism that involves the MRP1 chemotherapeutic drug resistance marker).

In some aspects of the invention, therapeutic agents are used to treat and/or prevent the development of a neoplasm in a patient by targeting the cell surface proteins listed in Table 1 with a protein-targeting agent. Non-limiting examples of targeting agents include antibodies, antibody fragments, inhibitors, nucleic acids, proteins, peptides, and peptidomimetic compounds. In other embodiments, the therapeutic agent is used to treat and/or prevent the development of a neoplasm by reducing the activity of a target protein. This can be accomplished by non-limiting means including preventing associations between proteins, preventing cell trafficking of proteins, tagging proteins for destruction, preventing expression of proteins, and inhibiting enzymatic reactions.

As used herein, the term “inhibitor” means a molecule that prevents a biomolecule, e.g., a protein, nucleic acid, lipid, carbohydrate, from initiating, taking part in, and/or completing a reaction. An inhibitor can stop a reaction by competitive, uncompetitive, or non-competitive means. Exemplary inhibitors include, but are not limited to, nucleic acids, proteins, small molecules, chemicals, peptides, peptidomimetic compounds, and analogs that mimic the binding site of an enzyme. In some embodiments, the inhibitor can be nucleic acid molecules including, but not limited to, siRNA that reduce the amount of functional protein in a cell.

The therapeutic agents can be composed of multiple parts, herein termed “components.” For example, the therapeutic agents can have a cell-associating component. A useful cell-associating component is an antibody or binding fragment of an antibody such as Fv, F(ab′)2, F(ab), Dab, and SC-Mab that binds to cell surface expressed cancer cell markers such as Pgp-1, multidrug resistance protein 1 (“MRP1”), BIP, BRCP, HSC70, nucleophosmin, vimentin, and HSP90. The cell-associating component can also be a compound that binds to a cell marker such as, but not limited to, an inhibitor of a cancer cell marker, a peptide, a peptidomimetic, a ligand, or a small molecule. As long as the interaction of the cell-associating component allows for cancer cell-specific targeting of the therapeutic agents, a compound is useful as a cell-associating component. The therapeutic agents also can include a cell-internalization component that allows the therapeutic agents to enter into the cell. For example, a cell-internalization component can be an agent that allows for cell membrane fusion between the therapeutic agents and the cancer cell, such as a liposome or immunoliposome (see, e.g., Drummond, et al, (2005) Ann. Rev. Pharmacol. Toxicol. 45: 495-528).

The cell-internalization component can be a dendrimer conjugate, which is a spherical polymer (see, e.g., Tomalia, D. A., et al., (1990) Angew. Chem. Int. Ed. Engl. 29: 5305). Synthesis and utilization of dendrimers has been postulated in the art, and dendrimers have been utilized for chemotherapeutic drug targeting in vitro (see, e.g., P. Singh, et al., (1994) Clin. Chem. 40: 1845). The protein-specific targeting component should bind to a protein target or a portion of protein target so as to decrease the effective activity of the enzyme in the targeted cancer cell. The cell surface protein-specific targeting component can be a nucleic acid that hybridizes specifically to sequences encoding a protein target or a portion of a polypeptide. In other embodiments, the cell surface protein-specific targeting component is selected from the group consisting of peptides, peptidomimetic compounds, small molecules specifically designed to bind to a protein target, and inhibitors. The aforementioned compounds are not intended to limit the range of compounds that can serve as the cell surface protein-specific targeting component, but are merely illustrative examples.

Moreover, cell surface protein-binding components can be composed of inhibitors. Alternatively, the therapeutic agents is an interfering RNA (RNAi) that specifically hybridizes to a segment or region of nucleic acids that express the proteins listed in Table 1 in the cancer cells. Ribonucleic acids used in RNAi to hybridize to target sequences can be of lengths between 10 to 20 bases, between 9 to 21 bases, between 7 to 23 bases, between 5 to 25 bases, between 25 to 35 bases, between 27 to 33 bases, and between 35 to 40 bases.

Following or at the time of treatment of a patient with targeted therapy, chemotherapeutic treatment is administered. Non-limiting examples of useful chemotherapeutic drugs for treating a patient include Actinomycin, Adriamycin, Altretamine, Asparaginase, Bleomycin, Busulfan, Capecitabine, Carboplatin, Carmustine, Chlorambucil, Cisplatin, Cladribine, Cyclophosphamide, Cytarabine, Dacarbazine, Dactinomycin, Daunorubicin, Docetaxel, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastine, Vincristine, and Vinorelbine. These drugs are commercially obtainable, e.g., from ScienceLab.com, Inc. (Kingwood, Tex.). Physician administered treatment with these chemotherapeutic drugs is well known in the art (see, e.g., Capers et al., (1993) Hosp. Pharm. 28(3):206-10).

Useful inhibitors are compounds that bind and reduce the “effective activity” of protein targets in a cell or cell sample. Compounds that reduce the effective activity of protein targets through binding to sites other than enzymatic regions include, but are not limited to, antibodies, antibody fragments such as “Fv,” “F(ab′)2,” “F(ab),” “Dab” and single chains representing the reactive portion of an antibody (“SC-Mab”), peptides, peptidomimetic compounds, and small molecules (see, e.g., Lopez-Alemany et al. (2003) Am. J. Hematol. 72(4): 234-42; Miles et al. (1991) Biochem. 30(6): 1682-91). The term “effective activity” as used herein refers to a protein's ability to perform a specific function at a level necessary to produce a phenotype such as chemotherapeutic drug resistance. The therapeutic agents can be specifically targeted to a neoplasm. To target the therapeutic agents, the agents can be incorporated into a liposome formulation, which can be an immunoliposome.

Typically, the therapeutic agents are targeted to the neoplasm orally, subcutaneously, transdermally, surgically, or intravenously. The therapeutic agents include, but are not limited to, compounds such as ligands, synthetic small molecules, nucleic acids, peptidomimetic compounds, inhibitors, peptides, proteins, and antibodies. The therapeutic agents can be an antibody or a binding fragment thereof. It should be noted that the nucleic acids can include, but are not limited to, DNA, RNA, RNA-DNA hybrids, siRNA, and aptamers. Moreover, the detectable label can be any label so long as the label does not affect the targeting function of the therapeutic agents. Labels include, but are not limited to, fluorophores, chemical dyes, radiolabels, chemiluminescent compounds, colorimetric enzymatic reactions, chemiluminescent enzymatic reactions, magnetic compounds, and paramagnetic compounds.

In addition, diagnostic assays for cell surface expression are useful for selecting patients in clinical studies potentially having neoplastic cells. Hence, the presence of certain proteins on the cell surface of cells identifies the patient as having a potential neoplasm, thereby allowing for alternative or additional prophylactic treatment and also for inclusion or exclusion from clinical studies. In certain aspects of the invention, antibodies targeted to the cell surface of certain tumor cells can block cell surface protein activity.

In some methods of the invention, a membrane fraction is isolated from a neoplastic cell sample prior to being contacted with therapeutic agents. The membrane fraction can be isolated using techniques known in the art. For instance, cell lysis can be accomplished by non-limiting techniques such as osmolysis, sonication, lysis by pressure means, or grinding of the cells by dounce. Cell lysis is typically followed by differential separation of cellular components using procedures known in the art (see, e.g., Neville (2005) J. Biophys. Biochem. Cytol. 8: 413-422). Purified membranes can be contacted with various therapeutic agents. These targeting agents include, but are not limited to, antibodies or binding fragments thereof, and inhibitors. Small molecules, peptides, and peptidomimetic compounds can also be used so long as these compounds show specific binding or association with the protein target. In addition, these compounds can be labeled for the purposes of detection as described below.

The invention also provides methods of treating or preventing the growth of a chemotherapeutic drug-resistant neoplasm in a patient in need thereof. The methods include administering an effective amount of therapeutic agents to a patient, the targeting agents being targeted to the neoplasm or to a site in close proximity to the neoplasm. Treatment of the patient includes administering a chemotherapeutic drug to kill the neoplastic cells after the cells have been targeted by the therapeutic agents to reduce or prevent the chemotherapeutic drug resistance of the neoplastic cells. Alternatively, the targeting agent and the chemotherapeutic drug can be administered simultaneously, e.g., as a single, linked therapeutic.

Other aspects of the invention provide a vaccine for the treatment and prevention of cancer in a patient in need thereof. As used herein, the term “vaccine” means any formulation or immunogen or antigen introduced into, such formulations include proteins, peptides, nucleic acids, peptidomimetic compounds, aptamers, and small molecules, the body for the specific purpose of generating a specific immune response in a patient. Vaccines have been used to treat disease conditions, typically occurring due to infection of particular viruses (see, e.g., Desombere et al. (2005) Clin. Exp. Immunol. 140(1):126-37). Recently, vaccines have been utilized to treat various forms of cancer (see, e.g., Nestle et al. (2005) Curr. Opin. Immunol. 17(2):163-9). Accordingly, vaccine formulations against particular cell surface proteins can be used to treat neoplasms arising from cancers such as a melanoma cell, a breast cancer cell, an ovarian cancer cell, a lung cancer cell, a lymphoma cell, a sarcoma cell, a leukemia cell, a retinoblastoma cell, a hepatoma cell, a myeloma cell, a glioma cell, a mesothelioma cell, a adenocarcinoma cell, and a carcinoma cell.

1.2 Therapeutic Agents

The present invention utilizes therapeutic agents for use in preventing or treating neoplasms. In some instances, therapeutic agents can be in the form of targeting agents that bind to cell surface proteins or portions of cell surface proteins. In some embodiments, useful targeting agents are proteins (hereinafter termed “protein-targeting agents”). As used herein, the term “protein-targeting agents” means a protein molecule, or polypeptide or peptide fragment thereof, that can interact, bind, or associate with a molecule or biological macromolecule in a sample. Such biological macromolecules include, but are not limited to, proteins, nucleic acids, simple carbohydrates, complex carbohydrates, fatty acids, lipoproteins, and/or triacylglycerides. Exemplary protein targeting agents include natural ligands of a receptor, hormones, antibodies, and portions thereof. The techniques associated with the binding of ligands and hormones to proteins as targeting agents have been demonstrated previously (see, e.g., Cutting et al., (2004) J. Biomol. NMR. 30(2): 205-10).

Protein targeting agents can be detectably labeled. As used herein, “detectably labeled” means that a targeting agent is operably linked to a moiety that is detectable. By “operably linked” is meant that the moiety is attached to the targeting agent by either a covalent or non-covalent (e.g., ionic) bond. Methods for creating covalent bonds are known (see, e.g., Wong, S. S., Chemistry of Protein Conjugation and Cross-Linking, CRC Press 1991; Burkhart et al., The Chemistry and Application of Amino Crosslinking Agents or Aminoplasts, John Wiley & Sons Inc., New York City, N.Y., 1999).

Useful labels can be, without limitation, fluorophores (e.g., fluorescein (FITC), phycoerythrin, rhodamine), chemical dyes, or compounds that are radioactive, chemoluminescent, magnetic, paramagnetic, promagnetic, or enzymes that yield a product that is colored, chemoluminescent, or magnetic. The signal is detectable by any suitable means, including spectroscopic, photochemical, biochemical, immunochemical, electrical, optical or chemical means. In certain cases, the signal is detectable by two or more means.

Labeled protein targeting agents allow detection of the level of expression of cell surface proteins in a cancer cell sample. For example, protein-targeting agents can be labeled for detection using chemiluminescent tags affixed to amino acid side chains. Useful tags include, but are not limited to, biotin, fluorescent dyes such as Cy5 and Cy3, and radiolabels (see, e.g., Barry and Soloviev (2000) Proteomics. 4(12): 3717-3726). Tags can be affixed to the amino terminal portion of a protein or the carboxyl terminal portion of a protein (see, e.g., Mattison and Kenney, (2002) J. Biol. Chem., 277(13): 11143-11148; Berne et al., (1990) J. Biol. Chem. 265(32): 19551-9). Indirect detection means can also be used to identify the cell markers. Exemplary but non-limiting means include detection of a primary antibody using a fluorescently labeled secondary antibody, or an antibody tagged with biotin such that it can be detected with fluorescently labeled streptavidin.

In some aspects, therapeutic agents can be in the form of nucleic acid targeting agents. As used herein, a “nucleic acid targeting agent” is defined as a nucleic acid capable of binding to a target nucleic acid of complementary sequence through one or more types of chemical bonds, usually through complementary base pairing, usually through hydrogen bond formation. Nucleic acid targeting agents include, but are not limited to, single-stranded RNA, double-stranded RNA, single-stranded DNA, double-stranded DNA, cDNA, cRNA, DNA-RNA hybrids, and aptamers. Single-stranded RNAs also include siRNA and antisense RNA. A nucleic acid targeting agent includes natural (i.e. A, G, U, C, or T) or modified (7-deazaguanosine, inosine, etc.) bases. In addition, the bases in targeting agents can be joined by a linkage other than a phosphodiester bond, so long as it does not interfere with hybridization. Thus, nucleic acid targeting agents can be peptide nucleic acids in which the constituent bases are joined by peptide bonds rather than phosphodiester linkages. The nucleic acid targeting agents can be prepared by converting the RNA to cDNA using known methods (see, e.g., Ausubel et. al., Current Protocols in Molecular Biology, Wiley 1999). The targeting agents can also be cRNA (see, e.g., Park et. al., (2004) Biochem. Biophys. Res. Commun. 325(4):1346-52).

Nucleic acid targeting agents can be produced from synthetic methods such as phosphoramidite methods, H-phosphonate methodology, and phosphite trimester methods. Nucleic acid targeting agents can also be produced by PCR methods. Such methods produce cDNA and cRNA sequences complementary to the mRNA. Such nucleic acid targeting agents can be detectably labeled, with, e.g., fluorophores (e.g., fluorescein (FITC), phycoerythrin, rhodamine), chemical dyes, or compounds that are radioactive, chemoluminescent, magnetic, paramagnetic, promagnetic, or enzymes that yield a product that is colored, chemoluminescent, or magnetic. The signal is detectable by any suitable means, including spectroscopic, photochemical, biochemical, immunochemical, electrical, optical or chemical means. In certain cases, the signal is detectable by two or more means. In certain embodiments, nucleic acid labels include fluorescent dyes, radiolabels, and chemiluminescent labels, which are examples that are not intended to limit the scope of the invention (see, e.g., Yu, et al., (1994) Nucleic Acids Res. 22(16): 3226-3232; Zhu, et al., (1994) Nucleic Acids Res. 22(16): 3418-3422).

Nucleic acid targeting agents can be detectably labeled using fluorescent labels. Non-limiting examples of fluorescent labels include 1- and 2-aminonaphthalene, p,p′diaminostilbenes, pyrenes, quaternary phenanthridine salts, 9-aminoacridines, p,p′-diaminobenzophenone imines, anthracenes, oxacarbocyanine, marocyanine, 3-aminoequilenin, perylene, bisbenzoxazole, bis-p-oxazolyl benzene, 1,2-benzophenazin, retinol, bis-3-aminopridinium salts, hellebrigenin, tetracycline, sterophenol, benzimidazolyl phenylamine, 2-oxo-3-chromen, indole, xanthen, 7-hydroxycoumarin, phenoxazine, salicylate, strophanthidin, porphyrins, triarylmethanes, flavin, xanthene dyes (e.g., fluorescein and rhodamine dyes); cyanine dyes; 4,4-difluoro-4-bora-3a,4a-diaza-s-indacene dyes and fluorescent proteins (e.g., green fluorescent protein, phycobiliprotein). These labels can be commercially obtained, e.g., from PerkinElmer Corp. (Boston, Mass.).

Other useful dyes are chemiluminescent dyes and can include, without limitation, biotin conjugated DNA nucleotides and biotin conjugated RNA nucleotides. Labeling of nucleic acid targeting agents can be accomplished by any means known in the art. (see, e.g., CyScribe™ First Strand cDNA Labeling Kit (#RPN6200, Amersham Biosciences, Piscataway, N.J.). The label can be added to the target nucleic acid(s) prior to, or after the hybridization. So called “direct labels” are detectable labels that are directly attached to, or incorporated into, the target nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid is biotinylated before the hybridization. After hybridization, an avidin-conjugated fluorophore binds the biotin bearing hybrid duplexes providing a label that is easily detected. (see, e.g., Laboratory Techniques in Biochemistry and Molecular Biology, Vol. 24: Hybridization With Nucleic Acid Targeting agents, P. Tijssen, ed. Elsevier, N.Y., (1993)).

The targeting agents of the present invention can also include inhibitors. Inhibitors can be labeled with detectable labels such as radiolabels, fluorescent labels, and chemiluminescent labels so long as the detectable label does not interfere with the binding or association of the inhibitor with its target compound (see, e.g., Singh and Wyeth (1991) Int. J. Rad. Appl. Instrum. [A]. 42(3):251-9).

1.3 Aptamers Directed Against Cell Surface Proteins

In addition, therapeutic agents can comprise aptamers. The term “aptamer,” used herein interchangeably with the term “nucleic acid ligand,” means a nucleic acid that, through its ability to adopt a specific three-dimensional conformation, binds to and has an antagonizing (i.e., inhibitory) effect on a target. The targets of the present invention are cell surface proteins, and hence the term cell surface aptamer or nucleic acid ligand is used. Inhibition of the target by the aptamer can occur by binding of the target, by catalytically altering the target, by reacting with the target in a way which modifies/alters the target or the functional activity of the target, by covalently attaching to the target as in a suicide inhibitor, by facilitating the reaction between the target and another molecule. Aptamers can be comprised of multiple ribonucleotide units, deoxyribonucleotide units, or a mixture of both types of nucleotide residues. In some embodiments, aptamers further comprise one or more modified bases, sugars or phosphate backbone units as described above.

Aptamers can be made by any known method of producing oligomers or oligonucleotides. Many synthesis methods are known in the art. For example, 2′-O-allyl modified oligomers that contain residual purine ribonucleotides, and bearing a suitable 3′-terminus such as an inverted thymidine residue (Ortigao et al., (1992) Antisense Res. Devel. 2:129-146) or two phosphorothioate linkages at the 3′-terminus to prevent eventual degradation by 3′-exonucleases, can be synthesized by solid phase beta-cyanoethyl phosphoramidite chemistry (Sinha et al., Nucleic Acids Res., 12:4539-4557 (1984)) on any commercially available DNA/RNA synthesizer. One method is the 2′-O-tert-butyldimethylsilyl (TBDMS) protection strategy for the ribonucleotides (Usman et al., (1987) J. Am. Chem. Soc., 109: 7845-7854), and all the required 3′-O-phosphoramidites are commercially available. In addition, aminomethylpolystyrene can be used as the support material due to its advantageous properties (McCollum and Andrus (1991) Tetrahedron Lett., 32:4069-4072). Fluorescein can be added to the 5′-end of a substrate RNA during the synthesis by using commercially available fluorescein phosphoramidites. In general, an aptamer oligomer can be synthesized using a standard RNA cycle. Upon completion of the assembly, all base labile protecting groups are removed by an eight hour treatment at 55° C. with concentrated aqueous ammonia/ethanol (3:1 v/v) in a sealed vial. The ethanol suppresses premature removal of the 2′-O-TBDMS groups that would otherwise lead to appreciable strand cleavage at the resulting ribonucleotide positions under the basic conditions of the deprotection (Usman et al., (1987) J. Am. Chem. Soc., 109: 7845-7854). After lyophilization, the TBDMS protected oligomer is treated with a mixture of triethylamine trihydrofluoride/triethylamine/N-methylpyrrolidinone for 2 hours at 60° C. to afford fast and efficient removal of the silyl protecting groups under neutral conditions (see Wincott et al., (1995) Nucleic Acids Res., 23:2677-2684). The fully deprotected oligomer can then be precipitated with butanol according to the procedure of Cathala and Brunel ((1990) Nucleic Acids Res., 18:201). Purification can be performed either by denaturing polyacrylamide gel electrophoresis or by a combination of ion-exchange HPLC (Sproat et al., (1995) Nucleosides and Nucleotides, 14:255-273) and reversed phase HPLC. For use in cells, synthesized oligomers are converted to their sodium salts by precipitation with sodium perchlorate in acetone. In certain embodiments, traces of residual salts are removed using small disposable gel filtration columns that are commercially available. As a final step the authenticity of the isolated oligomers can be checked by matrix assisted laser desorption mass spectrometry (Pieles et al., (1993) Nucleic Acids Res., 21:3191-3196) and by nucleoside base composition analysis.

Useful aptamers can also be produced through enzymatic methods, when the nucleotide subunits are available for enzymatic manipulation. For example, the RNA molecules can be made through in vitro RNA polymerase T7 reactions. They can also be made by strains of bacteria or cell lines expressing T7, and then subsequently isolated from these cells. As discussed below, the disclosed aptamers can also be expressed in cells directly using vectors and promoters.

In certain instances, the aptamers, like other nucleic acid molecules of the invention, further contain chemically modified nucleotides. One issue to be addressed in the diagnostic or therapeutic use of nucleic acids is the potential rapid degradation of oligonucleotides in their phosphodiester form in body fluids by intracellular and extracellular enzymes such as endonucleases and exonucleases before the desired effect is manifest. Certain chemical modifications of the nucleic acid ligand can be made to increase the in vivo stability of the nucleic acid ligand or to enhance or to mediate the delivery of the nucleic acid ligand (see, e.g., U.S. Pat. No. 5,660,985).

The stability of the aptamer can be greatly increased by the introduction of such modifications and as well as by modifications and substitutions along the phosphate backbone of the RNA. In addition, a variety of modifications can be made on the nucleobases themselves, which both inhibit degradation and which can increase desired nucleotide interactions or decrease undesired nucleotide interactions. Accordingly, once the sequence of an aptamer is known, modifications or substitutions can be made by the synthetic procedures described below or by procedures known to those of skill in the art.

Other modifications include the incorporation of modified bases (or modified nucleoside or modified nucleotides) that are variations of standard bases, sugars and/or phosphate backbone chemical structures occurring in ribonucleic (i.e., A, C, G and U) and deoxyribonucleic (i.e., A, C, G and T) acids. Included within this scope are, for example: Gm (2′-methoxyguanylic acid), Am (2′-methoxyadenylic acid), Cf (2′-fluorocytidylic acid), Uf (2′-fluorouridylic acid), Ar (riboadenylic acid). In some embodiments, the aptamers also include cytosine or any cytosine-related base including 5-methylcytosine, 4-acetylcytosine, 3-methylcytosine, 5-hydroxymethyl cytosine, 2-thiocytosine, 5-halocytosine (e.g., 5-fluorocytosine, 5-bromocytosine, 5-chlorocytosine, and 5-iodocytosine), 5-propynyl cytosine, 6-azocytosine, 5-trifluoromethylcytosine, N4, N4-ethanocytosine, phenoxazine cytidine, phenothiazine cytidine, carbazole cytidine or pyridoindole cytidine. The aptamer can further include guanine or any guanine-related base including 6-methylguanine, 1-methylguanine, 2,2-dimethylguanine, 2-methylguanine, 7-methylguanine, 2-propylguanine, 6-propylguanine, 8-haloguanine (e.g., 8-fluoroguanine, 8-bromoguanine, 8-chloroguanine, and 8-iodoguanine), 8-aminoguanine, 8-sulfhydrylguanine, 8-thioalkylguanine, 8-hydroxylguanine, 7-methylguanine, 8-azaguanine, 7-deazaguanine or 3-deazaguanine. The aptamer can still further include adenine or any adenine-related base including 6-methyladenine, N6-isopentenyladenine, N6-methyladenine, 1-methyladenine, 2-methyladenine, 2-methylthio-N6-isopentenyladenine, 8-haloadenine (e.g., 8-fluoroadenine, 8-bromoadenine, 8-chloroadenine, and 8-iodoadenine), 8-aminoadenine, 8-sulfhydryladenine, 8-thioalkyladenine, 8-hydroxyladenine, 7-methyladenine, 2-haloadenine (e.g., 2-fluoroadenine, 2-bromoadenine, 2-chloroadenine, and 2-iodoadenine), 2-aminoadenine, 8-azaadenine, 7-deazaadenine or 3-deazaadenine. Also included are uracil or any uracil-related base including 5-halouracil (e.g., 5-fluorouracil, 5-bromouracil, 5-chlorouracil, 5-iodouracil), 5-(carboxyhydroxylmethyl)uracil, 5-carboxymethylaminomethyl-2-thiouracil, 5-carboxymethylaminomethyluracil, dihydrouracil, 1-methylpseudouracil, 5-methoxyaminomethyl-2-thiouracil, 5′-methoxycarbonylmethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid, pseudouracil, 5-methyl-2-thiouracil, 2-thiouracil, 3-(3-amino-3-N-2-carboxypropyl)uracil, 5-methylaminomethyluracil, 5-propynyl uracil, 6-azouracil, or 4-thiouracil.

Examples of other modified base variants known in the art include, without limitation, e.g., 4-acetylcytidine, 5-(carboxyhydroxylmethyl) uridine, 2′-methoxycytidine, 5-carboxymethylaminomethyl-2-thioridine, 5-carboxymethylaminomethyluridine, dihydrouridine, 2′-O-methylpseudouridine, b-D-galactosylqueosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, b-D-mannosylqueosine, 5-methoxycarbonylmethyluridine, 5-methoxyuridine, 2-methylthio-N6-isopentenyladenosine, N-((9-b-D-ribofuranosyl-2-methylthiopurine-6-yl)carbamoyl)threonine, N-((9-b-D-ribofuranosylpurine-6-yl)N-methyl-carbamoyl)threonine, urdine-5-oxyacetic acid methylester, uridine-5-oxyacetic acid (v), wybutoxosine, pseudouridine, queosine, 2-thiocytidine, 5-methyl-2-thiouridine, 2-thiouridine, 4-thiouridine, 5-methyluridine, N-((9-b-D-ribofuranosylpurine-6-yl)carbamoyl)threonine, 2′-O-methyl-5-methyluridine, 2′-O-methyluridine, and wybutosine, 3-(3-amino-3-carboxypropyl)uridine.

Also included are the modified nucleobases described in U.S. Pat. Nos. 3,687,808, 3,687,808, 4,845,205, 5,130,302, 5,134,066, 5,175,273, 5,367,066, 5,432,272, 5,457,187, 5,459,255, 5,484,908, 5,502,177, 5,525,711, 5,552,540, 5,587,469, 5,594,121, 5,596,091, 5,614,617, 5,645,985, 5,830,653, 5,763,588, 6,005,096, and 5,681,941. Examples of modified nucleoside and nucleotide sugar backbone variants known in the art include, without limitation, those having, e.g., 2′ ribosyl substituents such as F, SH, SCH3, OCN, Cl, Br, CN, CF₃, OCF₃, SOCH₃, SO₂, CH₃, ONO₂, NO₂, N₃, NH₂, OCH₂CH₂OCH₃, O(CH₂)₂ON(CH₃)₂, OCH₂OCH₂N(CH₃)₂, O(C1-10 alkyl), O(C₂-10 alkenyl), O(C2-10 alkynyl), S(C1-10 alkyl), S(C2-10 alkenyl), S(C2-10 alkynyl), NH(C1-10 alkyl), NH(C2-10 alkenyl), NH(C2-10 alkynyl), and O-alkyl-O-alkyl. Desirable 2′ ribosyl substituents include 2′-methoxy (2′-OCH₃), 2′-aminopropoxy (2′OCH₂CH₂CH₂NH₂), 2′-allyl (2′-CH₂—CH═CH₂), 2′-O-allyl (2′-O—CH₂—CH═CH₂), 2′-amino (2′-NH₂), and 2′-fluoro (2′-F). In some embodiments, the 2′-substituent is in the arabino (up) position or ribo (down) position.

The present invention also includes aptamers made up of nucleotides and/or nucleotide analogs such as described above, or a combination of both, or are oligonucleotide analogs. Some embodiments have aptamers that contain nucleotide analogs at positions, which do not affect the function of the oligomer to bind its target.

There are several techniques that can be adapted for refinement or strengthening of the nucleic acid ligands binding to a particular target molecule or the selection of additional aptamers. One technique, generally referred to as “in vitro genetics” (see Szostak (1992) TIBS, 19:89), involves isolation of aptamer antagonists by selection from a pool of random sequences. For example, the pool of nucleic acid molecules from which the disclosed aptamers are isolated includes invariant sequences flanking a variable sequence of approximately twenty to forty nucleotides. This method has been termed Selective Evolution of Ligands by Exponential Enrichment (SELEX). Compositions and methods for generating aptamer antagonists of the invention by SELEX and related methods are known in the art and taught in, for example, U.S. Pat. Nos. 5,475,096 and 5,270,163. The SELEX process in general is further described in, e.g., U.S. Pat. Nos. 5,668,264, 5,696,249, 5,670,637, 5,674,685, 5,723,594, 5,756,291, 5,811,533, 5,817,785, 5,958,691, 6,011,020, 6,051,698, 6,147,204, 6,168,778, 6,207,816, 6,229,002, 6,426,335, and 6,582,918.

Other modifications useful for producing aptamers of the invention are known to one of ordinary skill in the art. Such modifications can be made post-SELEX process (modification of previously identified unmodified ligands) or by incorporation into the SELEX process. It has been observed that aptamers, or nucleic acid ligands, in general, are most stable, and therefore efficacious when 5′-capped and 3′-capped in a manner which decreases susceptibility to exonucleases and increases overall stability.

Cell surface protein targeting agents are specifically targeted to a site or region of a neoplasm to prevent the reduction of protein target activity in normal cells of the patient. Targeting mechanisms include non-limiting techniques such as conjugating the protein targeting agent to an agent that binds preferentially to a cancer cell marker (hereinafter termed “cancer cell targeting agent”). Cancer cell targeting agents include, but are not limited to, antibodies or binding fragments thereof, nucleic acids, peptides, small molecules, and peptidomimetic compounds. Alternatively, cancer cell targeting agents and cell surface protein targeting agents can be conjugated to functionalized chemical groups on non-limiting examples of inert supports such as polyethylene glycol, glass, synthetic polymers such as polyacrylamide, polystyrene, polypropylene, polyethylene, or natural polymers such as cellulose, Sepharose, or agarose, or conjugates with enzymes. Chemical conjugation techniques are well known in the art. Non-limiting examples of cancer cell markers that can be used for targeting of cell surface protein targeting agent include Pgp-1, MRP1, BIP, BRCP, HSC70, nucleophosmin, vimentin, and HSP90.

Alternatively, the cell surface protein targeting agent can be targeted to a neoplasm through a variety of invasive procedures. In the context of the present embodiment, such procedures include catheterization through an artery of a patient and depositing a protein-targeting agent within the tumor site. A surgeon can also apply the cell surface protein-targeting agent to the neoplasm by making an incision into the patient at a site that allows access to the tumor for placement of the targeting agent into, onto, or in close proximity to, the tumor. In some instances, a subject can also be intubated with subsequent introduction of the targeting agent into the tumor site through the tube. In other embodiments, the targeting agent can be administered to a patient orally, subcutaneously, intramuscularly, intravenously, or interperitoneally.

The targeting agent can be incorporated into a liposome before it is used. The term “liposome”, as used herein, refers to an artificial phospholipid bilayer vesicle. The liposome formulation can be used to facilitate lipid bilayer fusion with a target cell, thereby allowing the contents of the liposome or proteins associated with its surface to be brought into contact with the neoplastic cell. Liposomes can have antibodies associated with their bilayers that allow binding to targets on the neoplastic cell surface (hereinafter termed “immunoliposome”). Antibodies for these cell markers can be obtained commercially (e.g., Research Diagnostics, Inc., Flanders, N.J.; and Abcam, Inc., Cambridge, Mass.). Non-limiting examples of neoplastic cell targets to which such antibodies are specifically directed include Pgp-1, MRP1, BIP, BRCP, HSC70, nucleophosmin, vimentin, and HSP90.

1.4 Antibodies Against Cell Surface Proteins

Aspects of the present invention utilize antibodies directed against the cell surface proteins shown in Table 1 for use in diagnosis, detection, and prevention of cancer cells. Aspects of the present invention utilize treating/preventing cancer in a patient. The antibodies of the present invention, both monoclonal and polyclonal, are available from several commercial sources (e.g., Santa Cruz Biotechnology, Santa Cruz, Calif.; and Biogenesis, Inc., Kingston, N.H.). Antibodies can be administered to a patient orally, subcutaneously, intramuscularly, intravenously, or interperitoneally.

Aspects of the invention also utilize polyclonal antibodies for the detection of the protein targets shown in Table 1 and/or the treatment/prevention of cancer in a patient. As used herein, the term “polyclonal antibodies” means a population of antibodies that can bind to multiple epitopes on an antigenic molecule. A polyclonal antibody is specific to a particular epitope on an antigen, while the entire pool of polyclonal antibodies can recognize different epitopes. In addition, polyclonal antibodies developed against the same antigen can recognize the same epitope on an antigen, but with varying degrees of specificity. Polyclonal antibodies can be isolated from multiple organisms including, but not limited to, rabbit, goat, horse, mouse, rat, and primates. Polyclonal antibodies can also be purified from crude serums using techniques known in the art (see, e.g., Ausubel, et al., Current Protocols in Molecular Biology, Vol. 1, pp. 4.2.1-4.2.9, John Wiley & Sons, Inc., 1996).

The term “monoclonal antibody”, as used herein, refers to an antibody obtained from a population of substantially homogenous antibodies, i.e., the individual antibodies comprising the population are identical except for possible naturally occurring mutations that may be present in minor amounts. Monoclonal antibodies are highly specific, being directed against a single antigenic site. By their nature, monoclonal antibody preparations are directed to a single specific determinant on the target. Novel monoclonal antibodies or fragments thereof mean in principle all immunoglobulin classes such as IgM, IgG, IgD, IgE, IgA, or their subclasses or mixtures thereof. Non-limiting examples of subclasses include the IgG subclasses IgG1, IgG2, IgG3, IgG2a, IgG2b, IgG3, or IgGM. The IgG subtypes IgG1/κ and IgG2b/κ are also included within the scope of the present invention.

The monoclonal antibodies herein include hybrid and recombinant antibodies produced by splicing a variable (including hypervariable) domain of an antibody with a constant domain (e.g., “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab)₂, and Fv), so long as they exhibit the desired biological activity. (See, e.g., U.S. Pat. No. 4,816,567; Mage and Lamoyi, in Monoclonal Antibody Production Techniques and Applications, (Marcel Dekker, Inc., New York 1987, pp. 79-97). Thus, the modified “monoclonal” indicates the character of the antibody as being obtained from a substantially homogeneous population of antibodies, and is not to be construed as requiring production of the antibody by any particular method. For example, the monoclonal antibodies to be used in accordance with the present invention can be made by the hybridoma method (see, e.g., Kohler and Milstein (1975) Nature 256:495) or can be made by recombinant DNA methods (U.S. Pat. No. 4,816,567). The monoclonal antibodies can also be isolated from phage libraries generated using the techniques described in the art (see, e.g., McCafferty et al. (1990) Nature 348:552-554).

Alternative methods for producing antibodies can be used to obtain high affinity antibodies. Antibodies can be obtained from human sources such as serum. Additionally, monoclonal antibodies can be obtained from mouse-human heteromyeloma cell lines by techniques known in the art (see, e.g., Kozbor (1984) J. Immunol. 133, 3001; Boerner et al., (1991) J. Immunol. 147:86-95). Methods for the generation of human monoclonal antibodies using phage display, transgenic mouse technologies, and in vitro display technologies are known in the art and have been described previously (see, e.g., Osbourn et al. (2003) Drug Discov. Today 8: 845-51; Maynard and Georgiou (2000) Ann. Rev. Biomed. Eng. 2: 339-76; U.S. Pat. Nos. 4,833,077; 5,811,524; 5,958,765; 6,413,771; and 6,537,809).

In some embodiments, antibodies are used to inhibit a target protein to decrease the “effective activity” of the enzyme in a targeted cell, thereby increasing the chemosensitivity of the cell to chemotherapeutic treatments (see Lopez-Alemany et al. (2003) Am. J. Hematol. 72(4): 234-42).

1.5 RNA With Cell Surface Markers

Aspects of the present invention further allow for the treatment of a patient with a neoplasm or, in some embodiments, for the treatment of chemotherapeutic drug-resistant neoplasms using RNA interference (“RNAi”). As used herein, the term “RNA interference” refers to the blocking or preventing of cellular production of a particular protein by stopping the mechanisms of translation using small RNAs that hybridize to complementary sequences in a target mRNA. Anti-sense RNA strategies utilize the single-stranded nature of mRNA in a cell to block or interfere with translation of the mRNA into a protein. Antisense technology has been the most commonly described approach in protocols to achieve gene-specific interference. For antisense strategies, stoichiometric amounts of single-stranded nucleic acid complementary to the messenger RNA for the gene of interest are introduced into the cell.

In certain embodiments, the RNA comprises one or more strands of polymerized ribonucleotide. It includes modifications to either the phosphate-sugar backbone or the nucleoside. For example, the phosphodiester linkages of natural RNA are modified to include at least one of a nitrogen or sulfur heteroatom. For example, structural groups are added to the ribosyl or deoxyribosyl unit of the nucleotide, such as a methyl or allyl group at the 2′-O position or a fluoro group that substitutes for the 2′-O group. In certain instances, the linking group, such as a phosphodiester, of the nucleic acid is substituted or modified, for example with methyl phosphonates or O-methyl phosphates. Bases and sugars can also be modified, as is known in the art. RNA can also be modified to include “peptide nucleic acids” in which native or modified nucleic acid bases are attached to a polyamide backbone. Modifications in RNA structure can be tailored to allow specific genetic inhibition while avoiding a general panic response in some organisms, which is generated by dsRNA. Likewise, bases can be modified to block the activity of adenosine deaminase. RNA can be produced enzymatically or by partial/total organic synthesis, any modified ribonucleotide can be introduced by in vitro enzymatic or organic synthesis.

Methods of using siRNA to inhibit gene expression are well known in the art (see e.g., U.S. Pat. No. 6,506,559). Typically, complementary RNA sequences that can hybridize to a specific region of the target RNA are introduced into the cell. RNA annealing to the target transcripts allows the internal machinery of the cell to cut the dsRNA sequences into short segments. Such mechanisms have been utilized in in vitro and in vivo studies of human genes (see, e.g., Mizutani et al. (2002) J. Biol. Chem. 277(18):15859-64; Wang et al. (2005) Breast Cancer Res. 7(2):R220-8). In particular, the c-myc gene was inhibited in MCF7 breast cancer cell lines using the RNA interference technique (see Wang et al. (2005) Breast Cancer Res. 7(2):R220-8).

Interfering RNAs can be obtained by any means known in the art. For example, they can be synthetically produced using the Expedite™ Nucleic Acid Synthesizer (Applied Biosystems, Foster City, Calif.) or other similar devices (see, e.g., Applied Biosystems, Foster City, Calif.). Synthetic oligonucleotides also can be produced using methods well known in the art such as phosphoramidite methods (see, e.g., Pan et. al., (2004) Biol. Proc. Online. 6:257-262), H-phosphonate methodology (see, e.g., Agrawal et. al., (1987) Tetrahedron Lett. 28(31): 3539-3542) and phosphite trimester methods (Finnan et al. (1980) Nucleic Acids Symp. Ser. (7): 133-45).

1.6 Liposomes Comprising Cell Surface Targeting Agents

Another strategy that is employed for delivery of cell surface protein targeting agents is the use of immunoliposomes. Immunoliposomes incorporate antibodies against tumor-associated antigens into liposomes, which carry the therapeutic agent or an enzyme that activates an otherwise inactive prodrug (see, e.g., Lasic et al. (1995) Science 267: 1275-76). Immunoliposomal drugs can be used to successfully target and enhance anti-cancer efficacy (see, e.g., Maruyama et al. (1990) J. Pharm. Sci. 74: 978-84); Maruyama et al. (1995) Biochim. Biophys. Acta 1234: 74-80; Otsubo et al. (1998) Antimicrob. Agents Chemother. 42: 40-44; Lopes de Menezes et al. (1998) Cancer Res. 58: 3320-30).

Therapeutic agents, such as those shown in Table 1, can be incorporated into the membrane of the liposome through mechanisms known in the art (see, e.g., Pakunlu et al. (2004) Cancer Res. 64(17): 6214-24; Shimizu et al. (2002) Biol. Pharm. Bull. 25(6): 783-6; Zheng and Tan (2004) World J. Gastroenterol. 10(17): 2563-6). In addition, therapeutic agents and/or cell targeting agents can be associated with the outside of a liposome through covalent linkages to PEG polymers (see, e.g., Medina et al. (2004) Curr. Pharm. Des. 10(24): 2981-9). Furthermore, therapeutic agents can be incorporated into the hydrated inner compartment of the liposome (see, e.g., Medina et al. (2004) Curr. Pharm. Des. 10(24): 2981-9). A combination of the above mentioned liposome delivery methods can be used in a therapeutic composition.

Alternatively, modified LDL is used as tumor-specific ligands in targeting liposomal formulations containing therapeutic agents. For example, folate-coupled liposomes can be used to target therapeutics to tumors, which overexpress the folate receptor (Lee and Low (1994) J. Biol. Chem. 269: 3198-204; Lee and Low (1995) Biochim. Biophys. Acta 1233: 134-44; Rui et al. (1998) J. Am. Chem. Soc. 120: 11213-18; Gabizon et al. (1999) Bioconj. Chem. 10: 289-98). Transferrin has been employed as a targeting ligand to direct liposomal drugs to various types of cancer cell in vivo (Ishida and Maruyama (1998) Nippon Rinsho 56: 657-62; Kirpotin et al. (1997) Biochem. 36: 66-75). PEG-immunoliposomes with anti-transferring antibodies coupled to the distal ends of the PEG preferentially associate with C6 glioma cells in vitro and significantly increased gliomal doxorubicin uptake after treatment with the tumor-specific long-circulating liposomes containing doxorubicin (Eavarone et al. (2000) J. Biomed. Mater. Res. 51: 10-14).

Methods of delivering chemotherapeutic drugs and siRNA in vivo are known in the art (see, e.g., Mewani et al. (2004) Int. J. Oncol. 24(5): 1181-8; Chien et al. (2005) Cancer Gene Ther. 12(3): 3221-8). Liposomes have also been used for the targeted delivery of chemotherapeutic drugs, toxins, and labels (see, e.g., Pakunlu et al. (2004) Cancer Res. 64(17): 6214-24; Shimizu et al. (2002) Biol. Pharm. Bull. 25(6): 783-6; Zheng and Tan (2004) World J. Gastroenterol. 10(17): 2563-6). Liposome formulations for the delivery of chemotherapeutics and siRNA can be obtained from commercial suppliers, e.g., Eurogentec, Ltd. (Southampton, Hampshire, UK). In addition, methods for producing liposome micelle/chemotherapeutic formulations are well known in the art. For example, therapeutic drug micelles can be formed by combining a therapeutic drug and a phosphatidyl glycerol lipid derivative (PGL derivative). Briefly, the therapeutic drug and PGL derivative are mixed in a range of 1:1 to 1:2.1 to form a therapeutic drug mixture. Alternatively, the range of therapeutic drug to PGL derivative is 1:1.2; or 1:1.4; or 1:1.5; or 1:1.6; or 1:1.8 or 1:1.9 or 1:2.0 or 1:2.1. The mixture is then combined with an effective amount of at least a 20% organic solvent such as an ethanol solution to form micelles containing the therapeutic drug. Methods for inclusion of an antibody or tumor targeting ligand into the micelle formulation to produce immunoliposomes are known in the art and described further below. For example, methods for preparation and use of immunoliposomes are described in U.S. Pat. Nos. 4,957,735, 5,248,590, 5,464,630, 5,527,528, 5,620,689, 5,618,916, 5,977,861, 6,004,534, 6,027,726, 6,056,973, 6,060,082, 6,316,024, 6,379,699, 6,387,397, 6,511,676 and 6,593,308.

As used herein, the term “phosphatidyl glycerol lipid derivative (PGL derivative)” is any lipid derivative having the ability to form micelles and have a net negatively charged head group. This includes but is not limited to dipalmitoyl phosphatidyl glycerol (DPPG), dimyristoyl phosphatidyl glycerol, and dicapryl phosphatidyl glycerol. In one aspect, phosphatidyl derivatives with a carbon chain of 10 to 28 carbons and having unsaturated side aliphatic side chain are within the scope of this invention. The complexing of a therapeutic drug with negatively-charged phosphatidyl glycerol lipids having variations in the molar ratio giving the particles a net positive (1:1) neutral (1:2) or slightly negative (1:2.1) charge will allow targeting of different tissues in the body after administration. However, complexing of a therapeutic drug with negatively charged PGL has been shown to enhance the solubility of the therapeutic drug in many instances, thus reducing the volume of the drug required for effective antineoplastic therapy. In addition, the complexing of a therapeutic drug and negatively charged PGL proceeds to very high encapsulation efficiency, thereby minimizing drug loss during the manufacturing process. These complexes are stable, do not form precipitates and retain therapeutic efficacy after storage at 4° C. for at least four months. In order to achieve maximum therapeutic efficacy by avoiding rapid clearance from the blood circulation by the reticuloendothelial system (RES), immunoliposomal drug formulations incorporate components such as polyethylene glycol (PEG) (see, e.g., Klibanov et al. (1990) FEBS Lett. 268: 235-7; Mayuryama et al. (1992) Biochim. Biophys. Acta 1128: 44-49; Allen et al. (1991) Biochim. Biophys. Acta 1066: 29-36). Long-circulating immunoliposomes can be classified into two types: those with antibodies coupled to a lipid head growth (Maruyama et al. (1990) J. Pharm. Sci. 74: 978-84); and those with antibodies coupled to the distal end of PEG (Maruyama et al. (1997) Adv. Drug Del. Rev. 24: 235-42). In certain instances, it is advantageous to place the tumor-specific antibodies at the distal end of the PEG polymer to obtain efficient target binding by avoiding steric hindrance from the PEG chains.

1.7 Vaccines Against Cell Surface Markers

The invention includes known methods of preparing and using tumor antigen vaccines for use in treating neoplasms, and treating chemotherapeutic drug-resistance in neoplasms. The invention also includes methods of preparing and using tumor antigen vaccines for use in preventing cancers or for use in preventing cancers from becoming chemotherapeutic drug-resistant. The vaccine can be made using polypeptides from the proteins listed in Table 1, or polypeptide or peptide fragments thereof, and at least one pharmaceutically acceptable carrier.

Vaccines can be made to prevent the development of neoplasms from cells including, but not limited to, melanoma cells, breast cancer cells, ovarian cancer cells, lung cancer cells, lymphoma cells, sarcoma cells, leukemia cells, retinoblastoma cells, hepatoma cells, myeloma cells, glioma cells, mesothelioma cells, adenocarcinoma cells, and carcinoma cells. In addition, these cells can be obtained from various tissues such as breast, skin, lymphatic, prostate, bone, blood, brain, liver, thymus, kidney, lung, and ovary.

For example, U.S. Pat. No. 6,562,347 which teaches the use of a fusion polypeptide including a chemokine and a tumor antigen which is administered as either a protein or nucleic acid vaccine to elicit an immune response effective in treating or preventing cancer. Chemokines are a group of usually small secreted proteins (7-15 kD) induced by inflammatory stimuli and are involved in orchestrating the selective migration, diapedesis and activation of blood-born leukocytes that mediate the inflammatory response (see Wallack (1993) Ann. NY Acad. Sci. 178). Chemokines mediate their function through interaction with specific cell surface receptor proteins. At least four chemokine subfamilies have been identified as defined by a cysteine signature motif, termed CC, CXC, C and CX3C, where C is a cysteine and X is any amino acid residue. Structural studies have revealed that at least both CXC and CC chemokines share very similar tertiary structure (monomer), but different quaternary structure (dimer). For the most part, conformational differences are localized to sections of loop or the N-terminus. In the instant invention, for example, a human BIP polypeptide sequence (such as that shown in Table 1), or polypeptide fragment thereof, and a chemokine sequence are fused together and used in an immunizing vaccine. The chemokine portion of the fusion can be a human monocyte chemotactic protein-3, a human macrophage-derived chemokine or a human SDF-1 chemokine. The cell surface protein portion of the fusion is a portion shown in routine screening to have a strong antigenic potential. Immunological compositions, including vaccines, and other pharmaceutical compositions containing the proteins shown in Table 1, or portions thereof, are used within the scope of the present invention. One or more of the proteins in Table 1, or active or antigenic fragments thereof, or fusion proteins thereof can be formulated and packaged, alone or in combination with other antigens, using methods and materials known to those skilled in the art for vaccines. In certain embodiments, the immunological response is used therapeutically or prophylactically and provides antibody immunity or cellular immunity, such as that produced by T lymphocytes.

To enhance immunogenicity in some instances, the proteins are conjugated to a carrier molecule. Suitable immunogenic carriers include proteins, polypeptides or peptides such as albumin, hemocyanin, thyroglobulin and derivatives thereof, particularly bovine serum albumin (BSA) and keyhole limpet hemocyanin (KLH), polysaccharides, carbohydrates, polymers, and solid phases. Other protein derived or non-protein derived substances are known to those skilled in the art. An immunogenic carrier typically has a molecular mass of at least 1 kD, greater than 10 kD. Carrier molecules often contain a reactive group to facilitate covalent conjugation to the hapten. The carboxylic acid group or amine group of amino acids or the sugar groups of glycoproteins are often used in this manner. Carriers lacking such groups can often be reacted with an appropriate chemical to produce them. An immune response is produced when the immunogen is injected into animals such as mice, rabbits, rats, goats, sheep, guinea pigs, chickens, and other animals, such as mice and rabbits. Alternatively, a multiple antigenic peptide comprising multiple copies of the protein or polypeptide, or an antigenically or immunologically equivalent polypeptide is sufficiently antigenic to improve immunogenicity without the use of a carrier.

In some embodiments, the proteins shown in Table 1 or portions thereof, such as consensus or variable sequence amino acid motifs, or combination of proteins are administered with an adjuvant in an amount effective to enhance the immunogenic response against the conjugate. One adjuvant widely used in humans is alum (aluminum phosphate or aluminum hydroxide). Saponin and its purified component Quil A, Freund's complete adjuvant and other adjuvants used in research and veterinary applications are also available. Chemically defined preparations such as muramyl dipeptide, monophosphoryl lipid A, phospholipid conjugates, encapsulation of the conjugate within a proteoliposome, , and encapsulation of the protein in lipid vesicles such as Novasome™ lipid vesicles (Micro Vescular Systems, Inc., Nashua, N.H.) have been described previously (Goodman-Snitkoff et al. (1991) J. Immunol. 147:410415; Miller et al. (1992) J. Exp. Med. 176:1739-1744).

The invention utilizes polypeptide fragments, or subsequences of the intact polypeptides shown in Table 1. Such polypeptide subsequences, or a corresponding nucleic acid sequence that encodes them in the case of DNA vaccines, are selected so as to be highly immunogenic. The principles of antigenicity for the purpose of producing vaccines apply also to the use of polypeptide sequences for use as immunogens for generating polyclonal and monoclonal antibodies for use in diagnostics and therapeutics described herein.

Furthermore, a suitable adjuvant is typically combined with the immunogenic compound of a vaccine. As used herein, “adjuvant” or “suitable adjuvant” describes a substance capable of being combined with a protein or polypeptide to enhance an immune response in a subject without deleterious effect on the subject. A suitable adjuvant can be, but is not limited to, for example, an immunostimulatory cytokine, SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Other suitable adjuvants are well known in the art and include QS-21, Freund's adjuvant (complete and incomplete), alum, aluminum phosphate, aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dip-almitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion. QS-21, similarly to alum, complete Freund's adjuvant, SAF, etc., can be administered within hours of administration of a fusion protein.

1.8 Therapies

The invention provides for treatment or prevention of neoplasms, tumors, or metastases, including chemotherapeutic drug-resistant forms, by the administration of therapeutically or prophylactically effective amounts of therapeutic agents directed to the target proteins shown in Table 1. Moreover, the present invention provides cell surface protein-based therapies directed to the treatment or prevention of neoplasms and/or neoplasms that develop chemotherapeutic drug-resistant cancer using inhibitors. Certain therapies are utilized to decrease the effective activity of target proteins, such as those shown in Table 1, in a cancer cell, thereby increasing the sensitivity of the neoplasm to chemotherapeutic drugs. Also, the neoplastic cell's angiogenic phenotype or metastatic phenotype can be treated using nucleic acids complementary to a cell surface protein coding sequence.

Examples of types of cancer and proliferative disorders to be treated with the therapeutic agents of the invention include, but are not limited to, leukemia (e.g., myeloblastic, promyelocytic, myelomonocytic, monocytic, erythroleukemia, chronic myelocytic (granulocytic) leukemia, and chronic lymphocytic leukemia), lymphoma (e.g., Hodgkin's disease and non-Hodgkin's disease), fibrosarcoma, myxosarcoma, liposarcoma, chondrosarcoma, osteogenic sarcoma, angiosarcoma, endotheliosarcoma, Ewing's tumor, colon carcinoma, pancreatic cancer, breast cancer, ovarian cancer, prostate cancer, squamous cell carcinoma, basal cell carcinoma, adenocarcinoma, renal cell carcinoma, hepatoma, Wilms' tumor, cervical cancer, uterine cancer, testicular tumor, lung carcinoma, small cell lung carcinoma, bladder carcinoma, epithelial carcinoma, glioma, astrocytoma, oligodendroglioma, melanoma, neuroblastoma, retinoblastoma, dysplasia and hyperplasia. In a particular embodiment, therapeutic compounds of the invention are administered to men with prostate cancer (e.g., prostatitis, benign prostatic hypertrophy, benign prostatic hyperplasia (BPH), prostatic paraganglioma, prostate adenocarcinoma, prostatic intraepithelial neoplasia, prostato-rectal fistulas, and atypical prostatic stromal lesions). The treatment and/or prevention of cancer, cancers that develop chemotherapeutic drug-resistance and/or metastatic cancer includes, but is not limited to, alleviating symptoms associated with cancer, the inhibition of the progression of cancer, the promotion of the regression of cancer, and the promotion of the immune response.

The therapeutic agents can be administered in combination with other types of cancer treatments (e.g., radiation therapy, chemotherapy, hormonal therapy, immunotherapy, and anti-angiogenesis agents and other anti-tumor agents). Furthermore, therapeutic agents, such as nucleic acids, antibodies, proteins, inhibitors, ligands, organic molecules, peptidomimetic compounds, peptides, aptamers, and small molecules, can be administered simultaneously to treat cancer. For instance, an antibody against vimentin can be used in combination with an inhibitor directed against nucleophosmin, and as aptamer against HSP27. Alternatively, therapeutic agents can be used sequentially to treat cancer. Chemotherapeutic anti-tumor agents can be used simultaneously with, or subsequent to, administration of therapeutic agents. Examples of anti-tumor agents include, but are not limited to, ifosfamide, paclitaxel, taxanes, topoisomerase I inhibitors (e.g., CPf-11, topotecan, 9-AC, and GG-211), gemcitabine, vinorelbine, oxaliplatin, 5-fluorouracil (5-FU), leucovorin, vinorelbine, Actinomycin, Adriamycin, Altretamine, Asparaginase, Bleomycin, Busulfan, Capecitabine, Carboplatin, Carmustine, Chlorambucil, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastine, Vincristine, Vinorelbine., and temodal. Therapeutic agents directed against the cell surface proteins listed in Table 1 can be administered to a patient for the prevention or treatment of chemotherapeutic drug resistance prior to (e.g., 1 min., 15 min., 30 min., 45 min., 1 hour, 2 hours, 4 hours, 6 hours, 8 hours, 12 hours, 24 hours, 2 days, or 1 week before), subsequent to (e.g., 1 min., 15 min., 30 min., 45 min., 1 hour, 2 hours, 4 hours, 6 hours, 8 hours, 12 hours, 24 hours, 2 days, or 1 week after), or concomitantly with the administration of the anti-tumor agent to the subject.

The cell surface protein-targeted therapeutics described herein, can be administered to a patient or a human in need thereof, for the prevention or treatment of chemotherapeutic drug resistance prior to (e.g., 1 min., 15 min., 30 min., 45 min., 1 hour, 2 hours, 4 hours, 6 hours, 8 hours, 12 hours, 24 hours, 2 days, or 1 week before), subsequent to (e.g., 1 min., 15 min., 30 min., 45 min., 1 hour, 2 hours, 4 hours, 6 hours, 8 hours, 12 hours, 24 hours, 2 days, or 1 week after), or concomitantly with the administration of chemotherapeutic drugs described herein. More specifically, nucleic acids complementary to mRNAs encoding the cell surface proteins listed in Table 1 and/or antibodies and/or inhibitors thereof, and/or peptidometric compounds and/or organic molecules are administered prior to (e.g., 1 min., 15 min., 30 min., 45 min., 1 hour, 2 hours, 4 hours, 6 hours, 8 hours, 12 hours, 24 hours, 2 days, or 1 week before), subsequent to (e.g., 1 min., 15 min., 30 min., 45 min., 1 hour, 2 hours, 4 hours, 6 hours, 8 hours, 12 hours, 24 hours, 2 days, or 1 week after), or concomitantly with, the administration of chemotherapeutic drugs. These therapeutic agents can be incorporated into a liposome for transport into a cell.

It should be noted that the therapies discussed above can be used in combination as well. In addition, a protein-targeting agent typically binds to one cell surface protein marker. For instance, a protein-targeting agent specific for the BIP protein marker binds to only the BIP protein marker. It, however, is possible for a protein-targeting agent to bind to multiple cell surface targets listed in Table 1 provided that the peptide sequences are sufficiently similar to one another.

Furthermore, peptidomimetic compounds, antibodies, antigen-binding fragments of antibodies, nucleic acids, organic molecules, peptides, proteins, small molecules, and inhibitors can be used simultaneously or sequentially in any combination so long as the various combinations do not interfere with one another during treatment. Also, any combination of therapeutic agents directed against any combination of cell surface proteins listed in Table 1 can be utilized simultaneously or sequentially.

The cell surface protein-targeted therapeutics can be administered by any mechanism known in the art such that therapeutics can contact the cancerous growth or tumor. For example, therapies can be administered during an open surgical procedure in which the physician places the therapy into direct contact with the tumor. Alternatively, therapies can be administered in the form of an aerosol or vapor through an inhaler. In other methods, a patient can be intubated, and the therapeutics placed into the patient through the tube. The above-described means are not meant to be limiting.

Therapy can consist of the administration of any combination of therapeutic agents directed against the surface protein markers listed in Table 1. Accordingly, useful therapeutic formulations can consist of one or more therapeutic agents directed against one or more proteins listed in Table 1.

Alternatively, at least two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, or thirty or more therapeutic agents, can be administered to a patient in need thereof.

To illustrate a potential combination of therapeutic agents to treat a patient suffering from cancer, a clinician can administer a siRNA directed against BIP as well as an antibody that binds to the PDI/ER-60 precursor protein. The clinician can inject the formulation into the patient in combination, or sequentially, with a chemotherapeutic agent such as vinblastin. Alternatively, a clinician can combine the above therapeutic agents, vinblastin, and an additional therapeutic agent, such as an inhibitor against another protein listed in Table 1, into a liposome formulation. The formulation can be administered nasally, orally, or by any other means to treat the patient.

To further illustrate the potential combinations of therapeutic agents, a clinician can administer anti-ASF-2 antibodies in combination with valosin containing protein-directed siRNA. This combination can be further combined with nm23-H1-directed siRNA, anti-thioredoxin peroxidase 4 antibodies, ubiquilin-1 siRNA, inhibitors of AU-rich element RNA-binding protein, and annexin-1 siRNA. The treatment can be incorporated into a pool of liposomes in which each liposome within the pool has a different complement of therapeutic agents. In some instances, therapeutic agents incorporated into liposomes can be used in combination with therapeutic agents that are “free” in solution. In all instances, the therapeutic agents can be used in combination with radiotherapy, chemotherapeutic drugs, hormonal therapy, immunotherapy, and anti-tumor agents. The preceding example is not intended to limit the scope of the present invention, but is merely meant to illustrate potential uses of the present invention.

In addition to the preceding examples, inhibitors of one or more cell surface proteins listed in Table 1 can be used in combination with one or more peptides directed to limiting the activity or expression of one or more cell surface proteins listed in Table 1, which can be further used in combination with one or more siRNA sequences directed to one or more cell surface proteins listed in Table 1. These combinations can be further combined with one or more antibodies directed against one or more cell surface proteins listed in Table 1. This therapeutic combination can be still further combined with one or more peptidomimetic compounds directed against one or more cell surface protein listed in Table 1. Such a therapeutic combination can be used with radiotherapy, chemotherapeutic drugs, hormonal therapy, immunotherapy, and anti-tumor agents.

Moreover, the therapeutic agents of the present invention can be used in combination with enhancers of the immune system. Within certain aspects of the present invention, one or more compounds can be a non-specific immune response enhancer. For example, a non-specific immune response enhancer is any substance that enhances an immune response to an exogenous antigen. Examples of non-specific immune response enhancers include adjuvants, biodegradable microspheres (e.g., polylactic galactide) and liposomes (into which the compound is incorporated; see e.g., Fullerton, U.S. Pat. No. 4,235,877). Immune response enhancers are further described in U.S. Pat. No. 6,962,980. The therapies of the present invention can be used simultaneously with immune enhancers or sequentially with immune enhancers.

1.8.1 Using Cell Surface Markers for Therapeutic Agent Targeting

The cell surface proteins listed in Table 1 can be further utilized for targeting of a therapeutic agent to a cancer cell. In these instances, targeting agents directed against the cell surface proteins listed in Table 1 can target certain other therapeutic agents to the cell. Examples of cell surface targeting agents include, but are not limited to, antibodies or antigen-binding fragments thereof, inhibitors, ligands, peptides, peptidomimetic compounds, polypeptides, nucleic acids, and small molecules. The cell surface targeting agents can be associated with a delivery vehicle such as a liposome, dendrimer, or PEGylated liposome to facilitate delivery of the vehicle to the neoplastic cells. For instance, any combination of targeting agents directed against any combination of cell surface proteins listed in Table 1 can be associated with the surface of a liposome through hydrophobic, covalent, or ionic interactions. In this example, the liposome can be loaded with a therapy such as radiotherapy, a chemotherapeutic drug, or an anti-tumor agent.

Furthermore, the present therapies can be used in combinations with other cancer treatments such as anti-angiogenesis therapy. Such anti-angiogenesis therapy includes, but is not limited to, administration of angiostatin, angiostatin fragments, angiostatin antisera, angiostatin receptor agonists or angiostatin receptor antagonists (see, e.g., U.S. Pat. No. 6,949,511). Such anti-angiogenesis treatments can be combined with pharmaceutically acceptable excipients, and optionally sustained-release compounds or compositions, such as biodegradable polymers, to form therapeutic compositions. Anti-angiogenesis treatments typically are administered as vaccines, inhibitors of known angiogenesis factors such bFGF (see, e.g., U.S. Pat. No. 6,949,511), siRNA treatments directed against known angiogenesis factors, and peptides or proteins that interact with known angiogenesis factors. The therapies of the present invention can be used simultaneously with anti-angiogenesis treatments or sequentially with anti-angiogenesis treatments.

In some embodiments, the cell surface-targeting agent is at least one component of a cell surface protein-targeted agent. As used herein, the term “cell surface protein-targeted agent” means a compound composed of at least one cell surface-binding component and at least one therapeutic component. The components of the cell surface protein-targeted agent can be associated by hydrogen bonds, covalent bonds, hydrophobic interactions, van der Waals forces, ionic bonds, and London forces. The components can be contained within a delivery vehicle such as a liposome, an immunoliposome, a dendrimer, and pegylated liposomes. As used herein, the term “cell surface-binding component” means a molecule or compound capable of interacting, binding, or associating with at least one cell surface protein listed in Table 1. Cell surface-binding components can bind, interact, or associate with target cell surface proteins through covalent bonding, hydrogen bonding, van der Waals forces, London forces, hydrogen bonding, and ionic bonding. Cell surface-binding components can be composed of ligands, antibodies or antigen-binding fragrnents thereof, nucleic acids, inhibitors, aptamers, peptides, proteins, peptidomimetic compounds, and small molecules.

As used herein, the term “therapeutic component” means a therapeutic agent that can render a neoplastic cell more sensitive to another chemotherapeutic treatment, render a neoplastic cell incapable of metastasizing or inducing angiogenesis, and/or kill a neoplastic cell directly. Examples of therapeutic agents that can be a therapeutic component of the present embodiment include, but are not limited to, immunotoxins, bacterial toxins, and plant toxins. Exemplary bacterial toxins include Pseudomonas exotoxin and diphtheria toxin, which are well suited to forming recombinant single-chain or double-chain fusion toxins. Plant toxins include ricin, abrin, pokeweed antiviral protein, saporin and gelonin, and have generally been connected to ligands by disulfide-bond chemistry. Immunotoxins can contain a ligand such as a growth factor, monoclonal antibody, or fragment of an antibody, which is connected to a protein toxin. In all instances described above, after the ligand subunit binds to the surface of the target cell, the molecule internalizes and the toxin kills the cell.

1.8.2 Targeted Radiotherapies Utilizing Cell Surface Protein Markers

Therapies can also include radiation therapies in which targeting agents specific for the cell surface markers listed in Table 1 target the radioactive substance to the neoplasm. In some embodiments, radioisotopes are used as cytotoxic agents for cell surface protein-targeted therapeutics. In certain embodiments, antibodies of the present invention are coupled to one or more therapeutic agents. Suitable agents in this regard include radionuclides. Suitable radionuclides include ⁹⁰Y, ¹²³I, ¹²⁵I, ¹³¹I, ¹⁸⁶Re, ¹⁸⁸Re, ²¹¹At, and ²¹²Bi. Carriers specific for radionuclide agents, to facilitate attachment to the cell surface protein-targeting agent, include radiohalogenated small molecules and chelating compounds. For example, U.S. Pat. No. 4,735,792 discloses representative radiohalogenated small molecules and their synthesis. A radionuclide chelate is formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis.

An ideal radioligand therapy agent would accumulate selectively in target cells. The effectiveness of radiotherapy is due to the destruction of dividing cells resulting from radiation-induced damage to cellular DNA (see, e.g., Bloomer et al., (1977) Current Topics in Radiation Research Quarterly 12:513-25). In both therapeutic and imaging applications, any unbound, circulating radioligand is rapidly cleared by excretory systems, which helps protect normal organs and tissues. The radioligand can also be degraded by body processes, which will increase the clearance of the free radioisotope (Wiseman et al. (1995) Sem. Nucl. Med. 25:272-278).

Radioisotopes most suitable for therapeutic treatment include Auger-electron-emitting radioisotopes, e.g. ¹²⁵I, ¹²³I, ¹²⁴I, ¹²⁹I, ¹³¹I, ¹¹¹In, ⁷⁷Br, and other radiolabeled halogens. The choice of a suitable radioisotope can be optimized based on a variety of factors including the type of radiation emitted, the emission energies, the distance over which energy is deposited, and the physical half-life of the radioisotope. In certain instances, the radioisotopes used are those having a radioactive half-life corresponding to, or longer than, the biological half-life of the vimentin-targeted therapeutic. For example, in certain examples the radioisotope has a half-life between about 1 hour and 60 days, between 5 hours and 60 days, more between 12 hours and 60 days. ¹²⁵I has an advantage over other emitters that produce high-energy gamma rays (i.e., ¹¹¹In and ¹³¹I which require inpatient hospitalization and isolation ¹²⁵I will allow the development of outpatient-based treatments due to the limited amounts of radiation that escapes the body.

Radiolabeled therapeutics have typically been administered by intravenous, bolus injection (see, e.g., Kalofonos et al., (1989) J. Nucl. Med. 30:163-645; Virgolini et al., (1994) New England J. Med. 331:1116-21; Wiseman et al., (1995) Sem. Nucl. Med. 25:272-78; Lamberts et al., (1990) New England J. Med. 323:126-49; Krenning et al. (1992) J. Nucl. Med. 33:652-58; Krenning et al. (1989) The Lancet 1989:242-244.

Therapies can also include radiation therapies in which the radioactive substance is incorporated into a bead or a microsphere (see, e.g., Zielinski et al. (1983) Int. J. Appl. Radiat. Isot. 34(9): 1343-50). In some embodiments, the bead or microsphere can be conjugated to at least one of the therapeutic agents described above. The therapeutic agents are directed to one or more of the cell surface proteins listed in Table 1. The therapeutic agents facilitate the targeting of the beads or microspheres to the target neoplastic cells, allowing for improved targeting and efficacy of such treatments. In some embodiments, biodegradable microspheres (e.g., polylactate polyglycolate) are employed as carriers for the pharmaceutical compositions of this invention. Suitable biodegradable microspheres are disclosed, for example, in U.S. Pat. Nos. 4,897,268 and 5,075,109. These microspheres can be labeled with a radioactive nuclide through conjugation techniques known in the art. The therapies of the present invention can be used simultaneously or sequentially with the radiotherapy treatments described above.

1.9 Pharmaceutical Formulations and Methods of Treatment

The present invention provides for both prophylactic and therapeutic methods of treating a subject having cancer. Administration of a prophylactic agent can occur prior to the manifestation of symptoms characteristic of the neoplasm, such that development of the neoplasm is prevented or, alternatively, delayed in its progression. In general, the prophylactic or therapeutic methods comprise administering to the subject an effective amount of a therapeutic agent. The therapeutic agent can be comprised of multiple components, one of which can be a protein-binding component. Protein binding components, as envisioned by some embodiments of the present invention, are capable of binding to at least one of the proteins shown in Table 1. For example, the therapeutic agents can bind to the cell surface proteins expressed on neoplastic cells. Such binding can prevent the activity of cell surface proteins bound by the therapeutic agents, which can render the cells more susceptible to chemotherapeutic treatment.

Cell surface proteins can be targeted to neoplastic cells using a variety of targeting means. In some instances, a cancer cell-targeting component can be an antibody that binds to a neoplastic cell marker. The cell surface proteins targeted by the therapeutic agents of the present invention can be targeted to the neoplastic cells by vimentin, nucleophosmin or HSC70 antibodies, for example. Examples of cancer cell targeting components include monoclonal anti-vimentin antibodies and fragments thereof. Subsequent to internalization into a neoplastic cell, therapeutic agents can be administered to a patient to kill the neoplastic cell. Examples of suitable chemotherapeutic drugs include traditional chemotherapeutic agents such as Actinomycin, Adriamycin, Altretamine, Asparaginase, Bleomycin, Busulfan, Capecitabine, Carboplatin, Carmustine, Chlorambucil, Cisplatin, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastine, Vincristine, and Vinorelbine.

For such therapy, the compounds of the invention can be formulated for a variety of loads of administration, including systemic and topical or localized administration. Techniques and formulations generally can be found in Remmington's Pharmaceutical Sciences, Meade Publishing Co., Easton, Pa. For systemic administration, injection occurs through various mechanisms, including intramuscular, intravenous, intraperitoneal, and subcutaneous (s.c.). For injection, the compounds of the invention can be formulated in liquid solutions, in physiologically compatible buffers such as Hank's solution or Ringer's solution. In addition, the compounds are formulated in solid form and redissolved or suspended immediately prior to use. Lyophilized forms are also included.

For oral administration, the pharmaceutical compositions takes the form of, for example, tablets or capsules prepared by conventional means with pharmaceutically acceptable excipients such as targeting agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). In certain embodiments, the tablets are coated by methods well known in the art. Liquid preparations for oral administration take the form of, for example, solutions, syrups or suspensions, or they are presented as a dry product for constitution with water or other suitable vehicle before use. Such liquid preparations are prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). In some embodiments, the preparations contain buffer salts, flavoring, coloring and sweetening agents as appropriate.

Exemplary preparations for oral administration are suitably formulated to give controlled release of the active compound. For buccal administration the compositions, for example, take the form of tablets or lozenges formulated in a conventional manner. For administration by inhalation, exemplary compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, an exemplary dosage unit is determined by providing a valve to deliver a metered amount. Exemplary capsules and cartridges of e.g., gelatin for use in an inhaler or insufflator are formulated containing a powder mix of the compound and a suitable powder base such as lactose or starch.

Exemplary compounds are formulated for parenteral administration by injection, e.g., by bolus injection or continuous infusion. Exemplary formulations for injection are presented in unit dosage form, e.g., in ampoules or in multi-dose containers, with an added preservative. The compositions, for example, take such forms as suspensions, solutions or emulsions in oily or aqueous vehicles, and contain formulatory agents such as suspending, stabilizing and/or dispersing agents. Alternatively, the active ingredient is in powder form for constitution with a suitable vehicle, e.g., sterile pyrogen-free water, before use. The compounds, for example, also formulated in rectal compositions such as suppositories or retention enemas, e.g., containing conventional suppository bases such as cocoa butter or other glycerides.

In addition to the formulations described previously, the compounds are formulated as a depot preparation. Such long acting formulations are administered by implantation (for example, subcutaneously or intramuscularly) or by intramuscular injection. Thus, for example, the compounds are formulated with suitable polymeric or hydrophobic materials (for example as an emulsion in an acceptable oil) or ion exchange resins, or as sparingly soluble derivatives, for example, as a sparingly soluble salt. Other suitable delivery systems include microspheres, which offer the possibility of local noninvasive delivery of drugs over an extended period of time. This technology utilizes microspheres of precapillary size which can be injected via a coronary catheter into any selected part of the e.g. heart or other organs without causing inflammation or ischemia. The administered therapeutic is slowly released from these microspheres and taken up by surrounding tissue cells (e.g. endothelial cells).

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents can be used to facilitate permeation. Examples of transmucosal administration are nasal sprays or using suppositories. For topical administration, the oligomers of the invention are formulated into ointments, salves, gels, or creams as generally known in the art. A wash solution can be used locally to treat an injury or inflammation to accelerate healing.

In clinical settings, a therapeutic and gene delivery system for a cell surface protein-targeted therapeutic can be introduced into a patient by any of a number of methods, each of which is familiar in the art. For instance, a pharmaceutical preparation of a cell surface protein-targeted therapeutic can be introduced systemically, e.g., by intravenous injection.

The pharmaceutical preparation of a cell surface protein-targeted therapeutic compound of the invention can consist essentially of the compound in an acceptable diluent, or can comprise a slow release matrix in which the gene delivery vehicle or compound is imbedded.

The compositions are, if desired, presented in a pack or dispenser device that contains one or more unit dosage forms containing the active ingredient. The pack, for example, comprises metal or plastic foil, such as a blister pack. Exemplary pack or dispenser devices are accompanied by instructions for administration.

To demonstrate the methods according to the invention, a vimentin targeting agent was prepared and tested for its ability to increase the sensitivity of various cancer cell samples to chemotherapeutic drugs such as taxol. In particular, the well-characterized ovarian tumor cell line SKOV3 was used to identify the effects of an antibody directed against vimentin protein (see Examples 1 to 37 below).

Mice carrying human SKOV3 tumors were divided into the treatment groups shown in Table 1.

TABLE 1

| Groups | Treatment Regime | Dose |
|---|---|---|
| 1 | Vehicle | Q7 D × 3 |
| 2 | ¹³¹I-Isotypic Control | 250 μCi, Q7 D × 3 |
| 3 | ^{13l}I-Anti-Vimentin Antibody | 250 μCi, Q7 D × 3 |
| 4 | Taxol | 7.5 mg/kg, Q7 D × 3 |
| 5 | ¹³¹I-Anti-Vimentin Antibody + Taxol | 250 μCi, Q1 D × 1 |
| | | 7.5 mg/kg, Q7 D × 3 |
| 6 | ¹³¹I-Anti-Vimentin Antibody + Taxol | 250 μCi, Q7 D × 3 |
| | | 7.5 mg/kg, Q7 D × 3 |
| 7 | Taxol | 10 mg/kg, Q7 D × 3 |
| 8 | ¹³¹I-Anti-Vimentin Antibody + Taxol | 250 μCi, Q1 D × 1 |
| | | 10 mg/kg, Q7 D × 3 |

Q1 D × 1 and Q7 D × 3 were the treatment schedules utilized

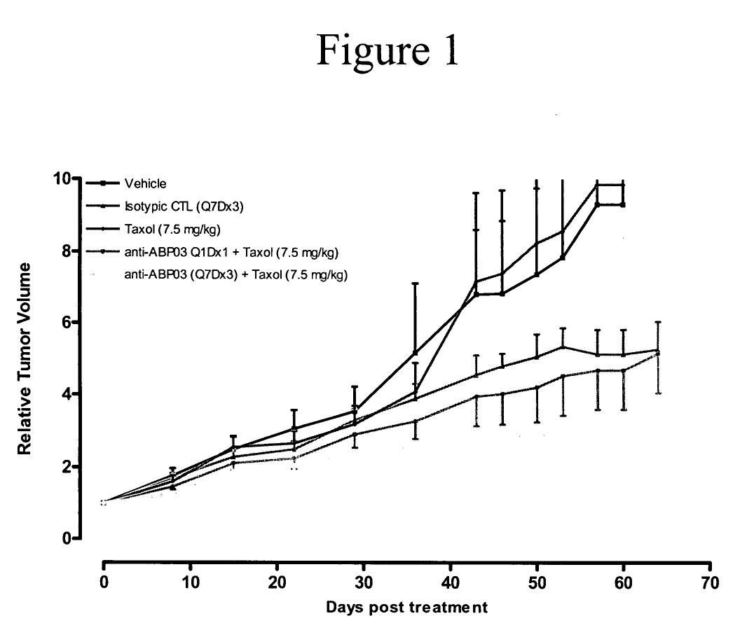

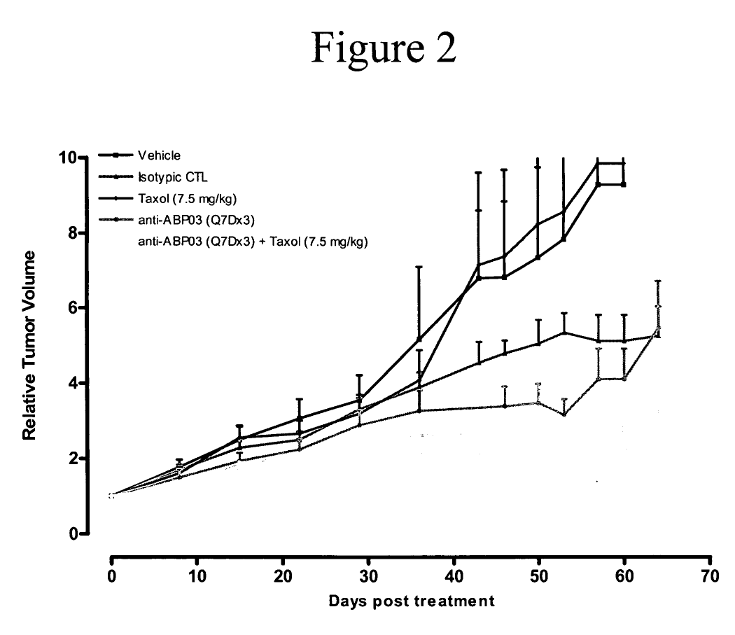

Relative tumor volumes for each group shown in Table 1 were determined after treatments were complete (FIGS. 1 and 2). Tumor volumes were greatest in mice treated with vehicle or isotypic controls. Specifically, mice treated with taxol and anti-vimentin antibodies had between 1.8-3 times less relative tumor volume than mice treated with vehicle or isotypic controls (FIGS. 1 and 2).

In addition, mice treated with anti-vimentin antibodies and taxol according to the Q7Dx3 schedule had smaller tumors than mice treated with taxol, alone, (FIGS. 1 and 2). When the Q1Dx1 treatment schedule was used for anti-vimentin antibody and taxol treatments, tumor volumes decreased significantly after 40 days of treatment up to 50 days of treatment (FIG. 2). It should be noted that mice treated with taxol, alone, had tumors that were smaller than tumors in mice treated with vehicle or isotypic control, but not as small as the tumors isolated from mice treated with anti-vimentin antibodies and taxol together (FIGS. 1 and 2). Therefore, the use of anti-vimentin antibodies in combination with taxol generates more effective treatment results than using taxol, alone.

### EXAMPLES

The invention will be further described in the following examples, which do not limit the scope of the invention described in the claims.

### Example 1

### Targeted Therapy Against SKOV3 Ovarian Cancer Cells Using Vimentin-Directed Therapy

1. Treatment of SKOV3 Cancer Cells

In order to determine whether therapeutic agents directed against the cell-surface markers described above were useful in treating a preexisting cancerous condition, thymic, nude mice, 5 to 7 weeks old, received an s.c. injection of the cells 5×10⁵ SKOV3 ovarian tumor cells, and tumors were allowed to form. Starting on the first day of treatment, tumor growth was measured by palpitation, and the volume of the xenograft was monitored every 4 days. Tumors were allowed to grow to a sufficient size (5.5 mm-100 mm) for appropriate analysis of the effects of vimentin-directed treatment on tumor sensitivity to chemotherapeutic drugs. Mice were then treated with vimentin-directed therapeutics. Mice in the combination therapy group were then treated with 250 μCi of ¹³¹I-anti-vimentin antibodies and taxol (either 7.5 mg/kg or 10 mg/kg daily). Control mice received vehicle, treatment with taxol, alone, (7.5 mg/kg or 10 mg/kg daily), or treatment with 250 μCi of ¹³¹I-anti-vimentin antibodies, alone. Taxol was obtained commercially from Sigma Corp. (St. Louis, Mo.).

All treatments were introduced by intravenous administration. Mice were then sacrificed and tumors excised for relative volume determinations.

As shown in FIGS. 1-2, mice treated with the combination of anti-vimentin antibodies and taxol had smaller tumors relative to mice treated with vehicle, taxol, alone, or anti-vimentin antibodies, alone. Overall, the relative volume of the tumors excised from mice treated with a combination of anti-vimentin antibodies and taxol decreased by up to 66% as compared to tumors excised from mice treated with vehicle.

### Example 2

### Vimentin/Taxol Liposome Formulation for Targeted Therapy

1. Treatment of SKOV3 Cancer Cells

In order to determine whether therapeutic agents directed against the cell-surface markers in Table 1 are useful in treating a preexisting cancerous condition, thymic, nude mice, 5 to 7 weeks old, receive an s.c. injection of the cells 5×10⁵ SKOV3 tumor cells, and tumors are allowed to form. Tumor growth starting on the first day of treatment is measured by palpitation and the volume of the xenograft is monitored every 4 days. Tumors are allowed to grow to a sufficient size (5.5 mm-100 mm) for appropriate analysis of the effects of vimentin/taxol combinatorial treatment on tumor volume. Mice are then treated with a liposome formulation containing anti-vimentin antibodies designed to decrease the level of expression of vimentin. The liposome formulation also contains taxol as a chemotherapeutic agent.

Liposome formulations are produced as described previously (Shi et al. (2000) Proc. Natl. Acad. Sci. USA. 97(13): 7567-7572). Briefly, POPC (19.2 μmol), DDAB (0.2 μmol), DSPE-PEG 2000 (0.6 μmol), and DSPE-PEG 2000-maleimide (30 nmol) are dissolved in chloroform/methanol (2:1, vol:vol) after a brief period of evaporation. The lipids are dispersed in 1 ml 0.05 M Tris-HCl buffer, pH 8.0, and are sonicated for 10 min. Anti-vimentin antibodies and taxol are added to the lipids. The liposome/anti-vimentin/taxol dispersion is evaporated to a final concentration of 200 mM at a volume of 100 μl. The dispersion is frozen in ethanol/dry ice for 4 to 5 min. The dispersion is then thawed at 40° C. for 1 to 2 min, and this freeze-thaw cycle is repeated 10 times. The liposome dispersion is diluted to a lipid concentration of 40 mM, is followed by extrusion 10 times each through two stacks each of 400 nm, 200 nm, 100 nm, and 50 nm pore size polycarbonate membranes, by using a hand held extruder (Avestin, Ottawa). The mean vesicle diameters are determined by quasielastic light scattering using a Microtrac Ultrafine Particle Analyzer (Leeds-Northrup, St. Petersburg, Fla.).

The liposome treatment introduces 250 μCi each of ¹³¹I-anti-vimentin antibodies into each mouse. Control mice receive vehicle, or receive treatment with liposomes loaded with taxol, alone, (7.5 mg/kg or 10 mg/kg daily) or treatment with liposomes loaded with ¹³¹I-anti-vimentin antibodies, alone.

A determination of decreased tumor size or cancer cell number is made by sacrificing the mice and excising the tumor. The size of the tumor in mice treated with the anti-vimentin antibodies and taxol is measured and compared to measurements obtained from tumors in mice treated with taxol, alone, or anti-vimentin antibody, alone. Tumor cell count is determined by trypsinizing tumors in DMEM medium supplemented with 10% fetal bovine serum until cells are in free suspension. Cells are then transferred to six well plates for counting. Cell counts are compared. All experiments are performed in triplicate.

The cancer cells treated with liposome/anti-vimentin antibodies/taxol treatment show an increase in sensitivity to the chemotherapeutic treatment regime. As a result, the mice that receive the composition show a better prognosis (i.e., smaller tumor or fewer tumor cells) as compared to mice that receive only liposomes loaded with taxol.

### Example 3

### Targeted Therapy Against SKOV3 Ovarian Cancer Cells Using Prohibitin and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 ovarian tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of prohibitin-directed treatment on tumor sensitivity to chemotherapeutic drugs. Mice are then treated with prohibitin-directed therapeutics. Mice in the combination therapy group are treated with 250 μCi of ¹³¹I-anti-prohibitin antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-prohibitin antibodies, alone. Taxol is obtained commercially from Sigma Corp. (St. Louis, Mo.). Anti-prohibitin antibodies are obtained commercially from EMD Biosciences, Inc. (San Diego, Calif.).

All treatments are introduced by intravenous administration. Mice are then sacrificed and tumors are excised for relative volume determinations. Relative tumor volume is determined as described above.

Tumors from mice treated with anti-prohibitin antibodies and taxol have decreased volume when compared to tumors from mice treated with taxol, alone, vehicle, or anti-prohibitin antibody, alone.

### Example 4

### Targeted Therapy Against SKOV3 Cancer Cells Using Nucleophosmin and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of nucleophosmin-directed treatment on tumor sensitivity to chemotherapeutic drugs. Mice are then treated with nucleophosmin-directed therapeutics. Mice in the combination therapy group are treated with 250 μCi of ¹³¹I-anti-nucleophosmin antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-nucleophosmin antibodies, alone. Taxol is obtained commercially from Sigma Corp. (St. Louis, Mo.). Anti-nucleophosmin antibodies are obtained commercially from Abcam, Inc. (Cambridge, Mass.).

All treatments are introduced by intravenous administration. Mice are then sacrificed and tumors are excised for relative volume determinations. Relative tumor volume is determined as described above.

Tumors from mice treated with anti-nucleophosmin antibodies and taxol have decreased volume when compared to tumors from mice treated with taxol, alone, vehicle, or anti-nucleophosmin antibody, alone.

### Example 5

### Targeted Therapy Against SKOV3 Cancer Cells Using HSC70 and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of HSC70-directed treatment on tumor sensitivity to chemotherapeutic drugs. Mice are then treated with HSC70-directed therapeutics. Mice in the combination therapy group are treated with 250 μCi of ¹³¹I-anti-HSC70 antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-HSC70 antibodies, alone. Taxol is obtained commercially from Sigma Corp. (St. Louis, Mo.). Anti-HSC70 antibodies are obtained commercially from Abcam, Inc. (Cambridge, Mass.).

All treatments are introduced by intravenous administration. Mice are then sacrificed and tumors are excised for relative volume determinations. Relative tumor volume is determined as described above.

Tumors from mice treated with anti-HSC70 antibodies and taxol have decreased volume when compared to tumors from mice treated with taxol, alone, vehicle, or anti-HSC70 antibody, alone.

### Example 6

### Targeted Therapy Against SKOV3 Cancer Cells Using BIP and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of BIP-directed treatment on tumor sensitivity to chemotherapeutic drugs. Mice are then treated with BIP-directed therapeutics. Mice in the combination therapy group are treated with 250 μCi of ¹³¹I-anti-BIP antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-BIP antibodies, alone. Taxol is obtained commercially from Sigma Corp. (St. Louis, Mo.). Anti-BIP antibodies are obtained commercially from BD Biosciences Pharmingen (San Jose, Calif.).

All treatments are introduced by intravenous administration. Mice are then sacrificed and tumors are excised for relative volume determinations. Relative tumor volume is determined as described above.

Tumors from mice treated with anti-BIP antibodies and taxol have decreased volume when compared to tumors from mice treated with taxol, alone, vehicle, or anti-BIP antibody, alone.

### Example 7

### Targeted Therapy Against SKOV3 Cancer Cells Using HSP60 and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of HSP60-directed treatment on tumor sensitivity to chemotherapeutic drugs. Mice are then treated with HSP60-directed therapeutics. Mice in the combination therapy group are treated with 250 μCi of ¹³¹I-anti-HSP60 antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-HSP60 antibodies, alone. Taxol is obtained commercially from Sigma Corp. (St. Louis, Mo.). Anti-HSP60 antibodies are obtained commercially from Abcam, Inc. (Cambridge, Mass.).

All treatments are introduced by intravenous administration. Mice are then sacrificed and tumors are excised for relative volume determinations. Relative tumor volume is determined as described above.

Tumors from mice treated with anti-HSP60 antibodies and taxol have decreased volume when compared to tumors from mice treated with taxol, alone, vehicle, or anti-HSP60 antibody, alone.

### Example 8

### Targeted Therapy Against SKOV3 Cancer Cells Using Annexin I and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of annexin I-directed treatment on tumor sensitivity to chemotherapeutic drugs. Mice are then treated with annexin I-directed therapeutics. Mice in the combination therapy group are treated with 250 μCi of ¹³¹I-anti-annexin I antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-annexin I antibodies, alone. Taxol is obtained commercially from Sigma Corp. (St. Louis, Mo.). Anti-annexin I antibodies are obtained commercially from Abcam, Inc. (Cambridge, Mass.).

All treatments are introduced by intravenous administration. Mice are then sacrificed and tumors are excised for relative volume determinations. Relative tumor volume is determined as described above.

Tumors from mice treated with anti-annexin I antibodies and taxol have decreased volume when compared to tumors from mice treated with taxol, alone, vehicle, or anti-annexin I antibody, alone.

### Example 9

### Targeted Theraipy Against SKOV3 Cancer Cells Using Ubiquilin 1 and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of ubiquilin 1-directed treatment on tumor sensitivity to chemotherapeutic drugs. Mice are then treated with ubiquilin 1-directed therapeutics. Mice in the combination therapy group are treated with 250 μCi of ¹³¹I-anti-ubiquilin 1 antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-ubiquilin 1 antibodies, alone. Taxol is obtained commercially from Sigma Corp. (St. Louis, Mo.). Anti-ubiquilin 1 antibodies are obtained commercially from Abnova Corp. (Taipei, Taiwan).

All treatments are introduced by intravenous administration. Mice are then sacrificed and tumors are excised for relative volume determinations. Relative tumor volume is determined as described above.

Tumors from mice treated with anti-ubiquilin 1 antibodies and taxol have decreased volume when compared to tumors from mice treated with taxol, alone, vehicle, or anti-ubiquilin 1 antibody, alone.

### Example 10

### Targeted Therapy Against SKOV3 Cancer Cells Using Thioredoxin Peroxidase 4 and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of thioredoxin peroxidase 4-directed treatment on tumor sensitivity to chemotherapeutic drugs. Mice are then treated with thioredoxin peroxidase 4-directed therapeutics. Mice in the combination therapy group are treated with 250 μCi of ¹³¹I-anti-thioredoxin peroxidase 4 antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-thioredoxin peroxidase 4 antibodies, alone. Taxol is obtained commercially from Sigma Corp. (St. Louis, Mo.). Anti-thioredoxin peroxidase 4 antibodies are obtained commercially from Abcam, Inc. (Cambridge, Mass.).

All treatments are introduced by intravenous administration. Mice are then sacrificed and tumors are excised for relative volume determinations. Relative tumor volume is determined as described above.

Tumors from mice treated with anti-thioredoxin peroxidase 4 antibodies and taxol have decreased volume when compared to tumors from mice treated with taxol, alone, vehicle, or anti-thioredoxin peroxidase 4 antibody, alone.

### Example 11

### Targeted Therapy Against SKOV3 Cancer Cells Using Cytokeratin 8 and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of cytokeratin 8-directed treatment on tumor sensitivity to chemotherapeutic drugs. Mice are then treated with cytokeratin 8-directed therapeutics. Mice in the combination therapy group are treated with 250 μCi of ¹³¹I-anti-cytokeratin 8 antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-cytokeratin 8 antibodies, alone. Taxol is obtained commercially from Sigma Corp. (St. Louis, Mo.). Anti-cytokeratin 8 antibodies are obtained commercially from Abcam, Inc. (Cambridge, Mass.).

All treatments are introduced by intravenous administration. Mice are then sacrificed and tumors are excised for relative volume determinations. Relative tumor volume is determined as described above.

Tumors from mice treated with anti-cytokeratin 8 antibodies and taxol have decreased volume when compared to tumors from mice treated with taxol, alone, vehicle, or anti-cytokeratin 8 antibody, alone.

### Example 12

### Targeted Therapy Against SKOV3 Cancer Cells Using PDI ER60 Precursor and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of PDI ER60 precursor-directed treatment on tumor sensitivity to chemotherapeutic drugs. Mice are then treated with PDI ER60 precursor-directed therapeutics. Mice in the combination therapy group are treated with 250 μCi of ¹³¹I-anti-PDI ER60 precursor antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-PDI ER60 precursor antibodies, alone. Taxol is obtained commercially from Sigma Corp. (St. Louis, Mo.). Anti-PDI ER60 precursor antibodies are obtained commercially from Sigma Corp. (St. Louis, Mo.).

All treatments are introduced by intravenous administration. Mice are then sacrificed and tumors are excised for relative volume determinations. Relative tumor volume is determined as described above.

Tumors from mice treated with anti-PDI ER60 precursor antibodies and taxol have decreased volume when compared to tumors from mice treated with taxol, alone, vehicle, or anti-PDI ER60 precursor antibody, alone.

### Example 13

### Targeted Therapy Against SKOV3 Cancer Cells Using HnRNPA/B and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of HnRNPA/B-directed treatment on tumor sensitivity to chemotherapeutic drugs. Mice are then treated with HnRNPA/B-directed therapeutics. Mice in the combination therapy group are treated with 250 μCi of ¹³¹I-anti-HnRNPA/B antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-HnRNPA/B antibodies, alone. Taxol is obtained commercially from Sigma Corp. (St. Louis, Mo.). Anti-HnRNPA/B antibodies are obtained commercially from Novus Biologicals, Inc. (Littleton, Colo.) and Cell Signaling Technology, Inc. (Danvers, Mass.).

All treatments are introduced by intravenous administration. Mice are then sacrificed and tumors are excised for relative volume determinations. Relative tumor volume is determined as described above.

Tumors from mice treated with anti-HnRNPA/B antibodies and taxol have decreased volume when compared to tumors from mice treated with taxol, alone, vehicle, or anti-HnRNPA/B antibody, alone.

### Example 14

### Targeted Therapy Against SKOV3 Cancer Cells Using HnRNPC and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of HnRNPC-directed treatment on tumor sensitivity to chemotherapeutic drugs. Mice are then treated with HnRNPC-directed therapeutics. Mice in the combination therapy group are treated with 250 μCi of ¹³¹I-anti-HnRNPC antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-HnRNPC antibodies, alone. Taxol is obtained commercially from Sigma Corp. (St. Louis, Mo.). Anti-HnRNPC antibodies are obtained commercially from ImmuQuest, Ltd. (Cleveland, UK).

All treatments are introduced by intravenous administration. Mice are then sacrificed and tumors are excised for relative volume determinations. Relative tumor volume is determined as described above.

Tumors from mice treated with anti-HnRNPC antibodies and taxol have decreased volume when compared to tumors from mice treated with taxol, alone, vehicle, or anti-HnRNPC antibody, alone.

### Example 15

### Targeted Therapy Against SKOV3 Cancer Cells Using HnRNPK and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of HnRNPK-directed treatment on tumor sensitivity to chemotherapeutic drugs. Mice are then treated with HnRNPK-directed therapeutics. Mice in the combination therapy group are treated with 250 μCi of ¹³¹I-anti-HnRNPK antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-HnRNPK antibodies, alone. Taxol is obtained commercially from Sigma Corp. (St. Louis, Mo.). Anti-HnRNPK antibodies are obtained commercially from ImmuQuest Ltd. (Cleveland, UK).

All treatments are introduced by intravenous administration. Mice are then sacrificed and tumors are excised for relative volume determinations. Relative tumor volume is determined as described above.

Tumors from mice treated with anti-HnRNPK antibodies and taxol have decreased volume when compared to tumors from mice treated with taxol, alone, vehicle, or anti-HnRNPK antibody, alone.

### Example 16

### Targeted Therapy Against SKOV3 Cancer Cells Using Rad 23 Homologue B and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of rad 23 homologue B-directed treatment on tumor sensitivity to chemotherapeutic drugs. Mice are then treated with rad 23 homologue B-directed therapeutics. Mice in the combination therapy group are treated with 250 μCi of ¹³¹I-anti-rad 23 homologue B antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-rad 23 homologue B antibodies, alone. Taxol is obtained commercially from Sigma Corp. (St. Louis, Mo.). Anti-rad 23 homologue B antibodies are obtained commercially from Abgent, Inc. (San Diego, Calif.).

All treatments are introduced by intravenous administration. Mice are then sacrificed and tumors are excised for relative volume determinations. Relative tumor volume is determined as described above.

Tumors from mice treated with anti-rad 23 homologue B antibodies and taxol have decreased volume when compared to tumors from mice treated with taxol, alone, vehicle, or anti-rad 23 homologue B antibody, alone.

### Example 17

### Targeted Therapy Against SKOV3 Cancer Cells Using ASF-2 and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of ASF-2-directed treatment on tumor sensitivity to chemotherapeutic drugs. Mice are then treated with ASF-2-directed therapeutics. Mice in the combination therapy group are treated with 250 μCi of ¹³¹I-anti-ASF-2 antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-ASF-2 antibodies, alone. Taxol is obtained commercially from Sigma Corp. (St. Louis, Mo.).

All treatments are introduced by intravenous administration. Mice are then sacrificed and tumors are excised for relative volume determinations. Relative tumor volume is determined as described above.

Tumors from mice treated with anti-ASF-2 antibodies and taxol have decreased volume when compared to tumors from mice treated with taxol, alone, vehicle, or anti-ASF-2 antibody, alone.

### Example 18

### Targeted Therapy Against SKOV3 Cancer Cells Using Tumor Protein D52-like 2 and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of tumor protein D52-like 2-directed treatment on tumor sensitivity to chemotherapeutic drugs. Mice are then treated with tumor protein D52-like 2-directed therapeutics. Mice in the combination therapy group are treated with 250 μCi of ¹³¹I-anti-tumor protein D52-like 2 antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-tumor protein D52-like 2 antibodies, alone. Taxol is obtained commercially from Sigma Corp. (St. Louis, Mo.). Anti-tumor protein D52-like 2 antibodies are obtained commercially from Abnova Corp. (Taipei, Taiwan).

All treatments are introduced by intravenous administration. Mice are then sacrificed and tumors are excised for relative volume determinations. Relative tumor volume is determined as described above.

Tumors from mice treated with anti-tumor protein D52-like 2 antibodies and taxol have decreased volume when compared to tumors from mice treated with taxol, alone, vehicle, or anti-tumor protein D52-like 2 antibody, alone.

### Example 19

### Targeted Therapy Against SKOV3 Cancer Cells Using Grp75 and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of Grp75-directed treatment on tumor sensitivity to chemotherapeutic drugs. Mice are then treated with Grp75-directed therapeutics. Mice in the combination therapy group are treated with 250 μCi of ¹³¹I-anti-Grp75 antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-Grp75 antibodies, alone. Taxol is obtained commercially from Sigma Corp. (St. Louis, Mo.). Anti-Grp75 antibodies are obtained commercially from Abcam, Inc. (Cambridge, Mass.).

All treatments are introduced by intravenous administration. Mice are then sacrificed and tumors are excised for relative volume determinations. Relative tumor volume is determined as described above.

Tumors from mice treated with anti-Grp75 antibodies and taxol have decreased volume when compared to tumors from mice treated with taxol, alone, vehicle, or anti-Grp75 antibody, alone.

### Example 20

### Targeted Therapy Against SKOV3 Cancer Cells Using ERp29 and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of ERp29-directed treatment on tumor sensitivity to chemotherapeutic drugs. Mice are then treated with ERp29-directed therapeutics. Mice in the combination therapy group are treated with 250 μCi of ¹³¹I-anti-ERp29 antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-ERp29 antibodies, alone. Taxol is obtained commercially from Sigma Corp. (St. Louis, Mo.). Anti-ERp29 antibodies are obtained commercially from Abcam, Inc. (Cambridge, Mass.).

All treatments are introduced by intravenous administration. Mice are then sacrificed and tumors are excised for relative volume determinations. Relative tumor volume is determined as described above.

Tumors from mice treated with anti-ERp29 antibodies and taxol have decreased volume when compared to tumors from mice treated with taxol, alone, vehicle, or anti-ERp29 antibody, alone.

### Example 21

### Targeted Therapy Against SKOV3 Cancer Cells Using HSP27 and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of HSP27-directed treatment on tumor sensitivity to chemotherapeutic drugs. Mice are then treated with HSP27-directed therapeutics. Mice in the combination therapy group are treated with 250 μCi of ¹³¹I-anti-HSP27 antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-HSP27 antibodies, alone. Taxol is obtained commercially from Sigma Corp. (St. Louis, Mo.). Anti-HSP27 antibodies are obtained commercially from Abcam, Inc. (Cambridge, Mass.).

All treatments are introduced by intravenous administration. Mice are then sacrificed and tumors are excised for relative volume determinations. Relative tumor volume is determined as described above.

Tumors from mice treated with anti-HSP27 antibodies and taxol have decreased volume when compared to tumors from mice treated with taxol, alone, vehicle, or anti-HSP27 antibody, alone.

### Example 22

### Targeted Therapy Against SKOV3 Cancer Cells Using Nm23-H1 and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of nm23-H1-directed treatment on tumor sensitivity to chemotherapeutic drugs. Mice are then treated with nm23-H1-directed therapeutics. Mice in the combination therapy group are treated with 250 μCi of ¹³¹I-anti-nm23-H1 antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-nm23-H1 antibodies, alone. Taxol is obtained commercially from Sigma Corp. (St. Louis, Mo.). Anti-nm23-H1 antibodies are obtained commercially from Abcam, Inc. (Cambridge, Mass.).

All treatments are introduced by intravenous administration. Mice are then sacrificed and tumors are excised for relative volume determinations. Relative tumor volume is determined as described above.

Tumors from mice treated with anti-nm23-H1 antibodies and taxol have decreased volume when compared to tumors from mice treated with taxol, alone, vehicle, or anti-nm23-H1 antibody, alone.

### Example 23

### Targeted Therapy Against SKOV3 Cancer Cells Using Valosin Containing Protein and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of valosin containing protein-directed treatment on tumor sensitivity to chemotherapeutic drugs. Mice are then treated with valosin containing protein-directed therapeutics. Mice in the combination therapy group are treated with 250 μCi of ¹³¹I-anti-valosin containing protein antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-valosin containing protein antibodies, alone. Taxol is obtained commercially from Sigma Corp. (St. Louis, Mo.). Anti-valosin containing protein antibodies are obtained commercially from Nanoprobes, Inc. (Yaphank, N.Y.).

All treatments are introduced by intravenous administration. Mice are then sacrificed and tumors are excised for relative volume determinations. Relative tumor volume is determined as described above.

Tumors from mice treated with anti-valosin containing protein antibodies and taxol have decreased volume when compared to tumors from mice treated with taxol, alone, vehicle, or anti-valosin containing protein antibody, alone.

### Example 24

### Targeted Therapy Against SKOV3 Cancer Cells Using 24.1D5 Antigen and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of 24.1D5 antigen-directed treatment on tumor sensitivity to chemotherapeutic drugs. Mice are then treated with 24.1D5 antigen-directed therapeutics. Mice in the combination therapy group are treated with 250 μCi of ¹³¹I-anti-24.1D5 antigen antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-24.1D5 antigen antibodies, alone. Taxol is obtained commercially from Sigma Corp. (St. Louis, Mo.).

All treatments are introduced by intravenous administration. Mice are then sacrificed and tumors are excised for relative volume determinations. Relative tumor volume is determined as described above.

Tumors from mice treated with anti-24.1D5 antigen antibodies and taxol have decreased volume when compared to tumors from mice treated with taxol, alone, vehicle, or anti-24.1D5 antigen antibody, alone.

### Example 25

### Targeted Therapy Against SKOV3 Cancer Cells Using AU-rich Element RNA Binding Protein and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of AU-rich element RNA binding protein-directed treatment on tumor sensitivity to chemotherapeutic drugs. Mice are then treated with AU-rich element RNA binding protein-directed therapeutics. Mice in the combination therapy group are treated with 250 μCi of ¹³¹I-anti-AU-rich element RNA binding protein antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-AU-rich element RNA binding protein antibodies, alone. Taxol is obtained commercially from Sigma Corp. (St. Louis, Mo.). Anti-AU-rich element RNA binding protein antibodies are obtained commercially from United States Biological (Swampscott, Mass.).

All treatments are introduced by intravenous administration. Mice are then sacrificed and tumors are excised for relative volume determinations. Relative tumor volume is determined as described above.

Tumors from mice treated with anti-AU-rich element RNA binding protein antibodies and taxol have decreased volume when compared to tumors from mice treated with taxol, alone, vehicle, or anti-AU-rich element RNA binding protein antibody, alone.

### Example 26

### Targeted Therapy Against SKOV3 Cancer Cells Using TCP-1ε and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of TCP-1ε-directed treatment on tumor sensitivity to chemotherapeutic drugs. Mice are then treated with TCP-1ε-directed therapeutics. Mice in the combination therapy group are treated with 250 μCi of ¹³¹I-anti-TCP-1ε antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-TCP-1ε antibodies, alone. Taxol is obtained commercially from Sigma Corp. (St. Louis, Mo.). Anti-TCP-1ε antibodies are obtained commercially from Abnova Corp. (Taipei, Taiwan).

All treatments are introduced by intravenous administration. Mice are then sacrificed and tumors are excised for relative volume determinations. Relative tumor volume is determined as described above.

Tumors from mice treated with anti-TCP-1ε antibodies and taxol have decreased volume when compared to tumors from mice treated with taxol, alone, vehicle, or anti-TCP-1ε antibody, alone.

### Example 27

### Targeted Therapy Against SKOV3 Cancer Cells Using β-tubulin and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of β-tubulin-directed treatment on tumor sensitivity to chemotherapeutic drugs. Mice are then treated with β-tubulin-directed therapeutics. Mice in the combination therapy group are treated with 250 μCi of ¹³¹I-anti-β-tubulin antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-β-tubulin antibodies, alone. Taxol is obtained commercially from Sigma Corp. (St. Louis, Mo.). Anti-β-tubulin antibodies are obtained commercially from Abcam, Inc. (Cambridge, Mass.).

All treatments are introduced by intravenous administration. Mice are then sacrificed and tumors are excised for relative volume determinations. Relative tumor volume is determined as described above.

Tumors from mice treated with anti-β-tubulin antibodies and taxol have decreased volume when compared to tumors from mice treated with taxol, alone, vehicle, or anti-β-tubulin antibody, alone.

### Example 28

### Targeted Therapy Against SKOV3 Cancer Cells Using β-actin and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of β-actin-directed treatment on tumor sensitivity to chemotherapeutic drugs. Mice are then treated with β-actin-directed therapeutics. Mice in the combination therapy group are treated with 250 μCi of ¹³¹I-anti-β-actin antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-β-actin antibodies, alone. Taxol is obtained commercially from Sigma Corp. (St. Louis, Mo.). Anti-β-actin antibodies are obtained commercially from Abcam, Inc. (Cambridge, Mass.).

All treatments are introduced by intravenous administration. Mice are then sacrificed and tumors are excised for relative volume determinations. Relative tumor volume is determined as described above.

Tumors from mice treated with anti-β-actin antibodies and taxol have decreased volume when compared to tumors from mice treated with taxol, alone, vehicle, or anti-β-actin antibody, alone.

### Example 29

### Targeted Therapy Against SKOV3 Cancer Cells Using γ-actin and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of γ-actin-directed treatment on tumor sensitivity to chemotherapeutic drugs. Mice are then treated with γ-actin-directed therapeutics. Mice in the combination therapy group are treated with 250 μCi of ¹³¹I-anti-γ-actin antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-γ-actin antibodies, alone. Taxol is obtained commercially from Sigma Corp. (St. Louis, Mo.). Anti-γ-actin antibodies are obtained commercially from Abnova Corp. (Taipei, Taiwan).

All treatments are introduced by intravenous administration. Mice are then sacrificed and tumors are excised for relative volume determinations. Relative tumor volume is determined as described above.

Tumors from mice treated with anti-γ-actin antibodies and taxol have decreased volume when compared to tumors from mice treated with taxol, alone, vehicle, or anti-γ-actin antibody, alone.

### Example 30

### Targeted Therapy Against SKOV3 Cancer Cells Using α-Internexin Therapy and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of α-internexin-directed treatment on tumor sensitivity to chemotherapeutic drugs. Mice are then treated with α-internexin-directed therapeutics. Mice in the combination therapy group are treated with 250 μCi of ¹³¹I-anti-α-internexin antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-α-internexin antibodies, alone. Taxol is obtained commercially from Sigma Corp. (St. Louis, Mo.). Anti-α-internexin antibodies are obtained commercially from Abcam, Inc. (Cambridge, Mass.).

All treatments are introduced by intravenous administration. Mice are then sacrificed and tumors are excised for relative volume determinations. Relative tumor volume is determined as described above.

Tumors from mice treated with anti-α-internexin antibodies and taxol have decreased volume when compared to tumors from mice treated with taxol, alone, vehicle, or anti-α-internexin antibody, alone.

### Example 31

### Targeted Therapy Against SKOV3 Cancer Cells Using Eukaryotic Elongation Factor 1δ Isoform 2 Therapy and Taxol Therapy

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of Eukaryotic Elongation Factor 1δ Isoform 2-directed treatment on tumor sensitivity to chemotherapeutic drugs. Mice are then treated with Eukaryotic Elongation Factor 1δ Isoform 2-directed therapeutics. Mice in the combination therapy group are treated with 250 μCi of ¹³¹I-anti-Eukaryotic Elongation Factor 1δ Isoform 2 antibodies and taxol (either 7.5 mg/kg or 10 mg/kg daily). Control mice receive vehicle, taxol, alone, (7.5 mg/kg or 10 mg/kg daily), or 250 μCi of ¹³¹I-anti-Eukaryotic Elongation Factor 1δ Isoform 2 antibodies, alone. Taxol is obtained commercially from Sigma Corp. (St. Louis, Mo.). Anti-Eukaryotic Elongation Factor 1δ Isoform 2 antibodies are obtained commercially from Abcam, Inc. (Cambridge, Mass.).

All treatments are introduced by intravenous administration. Mice are then sacrificed and tumors are excised for relative volume determinations. Relative tumor volume is determined as described above.

Tumors from mice treated with anti-Eukaryotic Elongation Factor 1δ Isoform 2 antibodies and taxol have decreased volume when compared to tumors from mice treated with taxol, alone, vehicle, or anti-Eukaryotic Elongation Factor 1δ Isoform 2 antibody, alone.

### Example 32

### Targeted Therapy Against Hematological Cancer Cells Using BIP and Prohibitin Combination Therapy

1. Treatment of Hematological Cancer Cells

In order to determine whether therapeutic agents directed against the proteins in Table 1 are useful in treating a preexisting cancerous condition, MHC-matched mice, 5 to 7 weeks old, receive an s.c. injection of the cells 5×10⁵ hematological tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of prohibitin-directed treatment on tumor sensitivity to chemotherapeutic drugs. Mice are then treated with a prohibitin siRNA and BIP siRNA (3 μg daily for each protein target fori 16 days) designed to decrease the level of expression of prohibitin and BIP. Control mice receive either no treatment, treatment with taxol or doxorubicin, alone, (4 mg/kg daily), or treatment with control siRNA sequences that are not complementary to murine prohibitin mRNA (3 μg daily for 16 days for each treatment) in combination with taxol or doxorubicin (4 mg/kg daily). All treatments are introduced by intravenous administration. Taxol and doxorubicin are obtained commercially from Sigma Corp. (St. Louis, Mo.).

Control siRNA sequences are utilized that do not represent binding sequences to murine prohibitin (3 μg daily for 16 days for each treatment). The animal's weight is measured every 4 days. Tumor growth starting on the first day of treatment is measured by palpitation and the volume of the xenograft is monitored every 4 days. The mice are anaesthetized and sacrificed when the mean tumor weight is over 1 g in the control group. Tumor tissue is excised from the mice and its weight is measured. Tumor weights from mice treated with the prohibitin and BIP siRNA and chemotherapeutic drugs are compared to tumor weights from mice treated with control siRNA and chemotherapeutic drugs. Tumor cell count is determined by trypsinizing tumors in DMEM medium supplemented with 10% fetal bovine serum until cells are in free suspension. Cells are then transferred to 6-well plates for counting. Cell counts are compared.

Treatment with siRNA specific for prohibitin mRNA sequences and BIP siRNA sequences increases the sensitivity of hematological tumors to chemotherapeutic drug treatment regimes. As a result, the mice that receive the composition show a better prognosis (i.e., smaller tumor or fewer tumor cells) as compared to mice that receive only the targeting agent or only the taxol or doxorubicin. Mice treated with the prohibitin/BIP siRNA combination therapy and chemotherapeutic drugs have fewer neoplastic cells than mice treated with control siRNA or chemotherapeutic drugs alone. All experiments are performed in triplicate.

2. Treatment of Mammary Adenocarcinoma

In further studies, the efficacy of combinatorial BIP-targeted and prohibitin-targeted therapeutic agents (siRNA) in treating mammary adenocarcinoma cells (MCF-7/AR) is assessed. Briefly, male thymic nude mice 5 to 7 weeks old, weighing 18 g to 22 g, are used for the MCF-7/ADR xenografts. Mice receive an s.c. injection of the cells using 5×10⁵ cells/inoculation under the shoulder. When the s.c. tumor is approximately 5.5 mm in size, mice are randomized into treatment groups of 4 including controls and groups receiving taxol or doxorubicin, alone (4 mg/kg), intraperitoneally (i.p.) every 2 days, prohibitin siRNA and BIP siRNA alone (3 μg daily for 16 days), or both taxol and prohibitin/BIP siRNA combinatorial therapy (3 μg of each siRNA daily for 16 days). Control siRNA sequences are utilized that do not represent binding sequences to murine prohibitin (3 μg daily for 16 days for each treatment). The animal's weight is measured every 4 days. Tumor growth starting on the first day of treatment is measured and the volume of the xenograft is monitored every 4 days. The mice are anaesthetized and sacrificed when the mean tumor weight is over 1 g in the control group. Tumor tissue is excised from the mice and its weight is measured. Tumor weights from mice treated with the prohibitin/BIP combinatorial therapy and chemotherapeutic drugs are compared to tumor weights from mice treated with control siRNA and chemotherapeutic drugs. Cell counts are compared. All experiments are performed in triplicate.

Mice treated with the prohibitin and BIP siRNA have smaller tumors by weight than mice treated with control siRNA. In addition, total cell numbers of tumors isolated from mice treated with prohibitin and BIP siRNA are lower than mice treated with control siRNA.

### Example 33

### Prohibitin Liposome Formulation for Targeted Therapy

1. Treatment of Hematological Cancer

In order to determine whether therapeutic agents directed against the proteins in Table 1 are useful in treating a preexisting cancerous condition, MHC-matched mice, 5 to 7 weeks old, receive an s.c. injection of the cells 5×10⁵ hematological tumor cells, and tumors are allowed to form. Tumor growth starting on the first day of treatment is measured by palpitation and the volume of the xenograft is monitored every 4 days. Tumors are allowed to grow to a sufficient size (5.5 mm) for appropriate analysis of the effects of prohibitin/BIP combinatorial treatment on tumor sensitivity to chemotherapeutic drugs. Mice are then treated with a liposome formulation containing prohibitin siRNA designed to decrease the level of expression of prohibitin.

Liposome formulations are produced as described previously (Shi et al. (2000) Proc. Natl. Acad. Sci. USA. 97(13): 7567-7572). Briefly, POPC (19.2 μmol), DDAB (0.2 μmol), DSPE-PEG 2000 (0.6 μmol), and DSPE-PEG 2000-maleimide (30 nmol) are dissolved in chloroform/methanol (2:1, vol:vol) after a brief period of evaporation. The lipids are dispersed in 1 ml 0.05 M Tris-HCl buffer, pH 8.0, and are sonicated for 10 min. Prohibitin siRNA and BIP siRNA are added to the lipids. The liposome/siRNA dispersion is evaporated to a final concentration of 200 mM at a volume of 100 μl. The dispersion is frozen in ethanol/dry ice for 4 to 5 min. The dispersion is then thawed at 40° C. for 1 to 2 min, and this freeze-thaw cycle is repeated 10 times. The liposome dispersion is diluted to a lipid concentration of 40 mM, is followed by extrusion 10 times each through two stacks each of 400 nm, 200 nm, 100 nm, and 50 nm pore size polycarbonate membranes, by using a hand held extruder (Avestin, Ottawa). The mean vesicle diameters are determined by quasielastic light scattering using a Microtrac Ultrafine Particle Analyzer (Leeds-Northrup, St. Petersburg, Fla.).

The liposome treatment introduces 3 μg each of prohibitin-targeted siRNA and BIP-targeted siRNA per day for 16 days. Control mice receive no treatment, or receive treatment with taxol or doxorubicin alone (4 mg/kg daily) or treatment with liposomes containing control siRNA sequences that are not complementary to murine prohibitin mRNA or BIP mRNA (3 μg daily for 16 days for each treatment) in combination with taxol or doxorubicin (4 mg/kg daily). Taxol and doxorubicin are obtained commercially from Sigma Corp. (St. Louis, Mo.).

A determination of decreased tumor size or cancer cell number is made by sacrificing the mice and excising the tumor. The size of the tumor in mice treated with the prohibitin and BIP therapeutic agents and chemotherapy is measured and compared to measurements obtained from tumors in mice treated with chemotherapy alone. Tumor cell count is determined by trypsinizing tumors in DMEM medium supplemented with 10% fetal bovine serum until cells are in free suspension. Cells are then transferred to six well plates for counting. Cell counts are compared. All experiments are performed in triplicate.

The cancer cells treated with liposome/prohibitin/BIP siRNA treatment show an increase in sensitivity to chemotherapeutic treatment regimes. As a result, the mice that receive the composition show a better prognosis (i.e., smaller tumor or fewer tumor cells) as compared to mice that receive only the targeting agent vincristin.

2. Treatment of Mammary Adenocarcinoma

In further studies, the efficacy of a prohibitin-targeted and BIP targeted combinatorial therapeutic treatments in treating a mammary adenocarcinoma cells (MCF-7) is assessed. Briefly, male thymic nude mice 5 to 7 weeks old, weighing 18 g to 22 g, are used for the MCF-7/ADR xenografts. Mice receive an s.c. injection of the cells using 5×10⁵ cells/inoculation under the shoulder.

Liposome formulations are produced as described previously (Shi et al. (2000) Proc. Natl. Acad. Sci. USA. 97(13): 7567-7572). Briefly, POPC (19.2 μmol), DDAB (0.2 μmol), DSPE-PEG 2000 (0.6 μmol), and DSPE-PEG 2000-maleimide (30 nmol) are dissolved in chloroform/methanol (2:1, vol:vol) after a brief period of evaporation. The lipids are dispersed in 1 ml 0.05 M Tris-HCl buffer, pH 8.0, and are sonicated for 10 min. Prohibitin siRNA and BIP siRNA are added to the lipids. The liposome/siRNA dispersion is evaporated to a final concentration of 200 mM at a volume of 100 μl. The dispersion is frozen in ethanol/dry ice for 4 to 5 min. The dispersion is then thawed at 40° C. for 1 to 2 min, and this freeze-thaw cycle is repeated 10 times. The liposome dispersion is diluted to a lipid concentration of 40 mM, is followed by extrusion 10 times each through two stacks each of 400 nm, 200 nm, 100 nm, and 50 nm pore size polycarbonate membranes, by using a hand held extruder (Avestin, Ottawa). The mean vesicle diameters are determined by quasielastic light scattering using a Microtrac Ultrafine Particle Analyzer (Leeds-Northrup, St. Petersburg, Fla.).

When the s.c. tumor is approximately 5.5 mm in size, mice are randomized into treatment groups of 4 including controls and groups receiving taxol or doxorubicin, alone (4 mg/kg), intraperitoneally (i.p.) every 2 days, prohibitin siRNA/liposome formulation alone (3 μg daily for 16 days), or both taxol and prohibitin and BIP siRNA/liposome formulation (3 μg daily for 16 days for each treatment). Control siRNA sequences are utilized that do not represent binding sequences to murine prohibitin (3 μg daily for 16 days for each treatment). The animal's weight is measured every 4 days. Tumor growth starting on the first day of treatment is measured and the volume of the xenograft is monitored every 4 days. The mice are anaesthetized and sacrificed when the mean tumor weight is over 1 g in the control group. Tumor tissue is excised from the mice and its weight is measured.

Mice treated with the prohibitin/BIP siRNA combination therapy have smaller tumors by weight than mice treated with control siRNA. In addition, total cell number in tumors isolated from mice treated with prohibitin/BIP siRNA combination therapy is lower than mice treated with control siRNA. Tumor weights from mice treated with the prohibitin/BIP siRNA combination therapies and chemotherapeutic drugs are compared to tumor weights from mice treated with control siRNA and chemotherapeutic drugs.

### Example 34

### Targeted Therapy Against SKOV3 Cells Using Vimentin, Prohibitin, and Nucleophosmin Therapy in Combination with Taxol

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of cell surface marker-directed treatment on tumor sensitivity to chemotherapeutic drugs. Mice are then treated with a combinatorial therapy in which antibodies directed against vimentin, prohibitin, and nucleophosmin are injected into mice having the SKOV3 tumors. Mice in the combinatorial therapy group are treated with 250 μCi each of: ¹³¹I-anti-vimentin antibodies, ¹³¹I-anti-prohibitin antibodies, and ¹³¹I-anti-nucleophosmin antibodies. Taxol is also administered to the combinatorial therapy group at concentrations of either 7.5 mg/kg or 10 mg/kg daily. Control mice receive vehicle, taxol, alone, (7.5 mg/kg or 10 mg/kg daily), 250 μCi of ¹³¹I-anti-vimentin antibodies, alone, 250 μCi of ¹³¹I-anti-nucleophosmin antibodies, alone, or 250 μCi of ¹³¹I-anti-prohibitin antibodies, alone.

All treatments are introduced by intravenous administration. Mice are then sacrificed and tumors are excised for relative volume determinations. Relative tumor volume is determined as described above.

Tumors from mice treated with the combination of anti-vimentin, anti-nucleophosmin, and anti-prohibitin antibodies in combination with taxol have decreased volume when compared to tumors from mice treated with taxol, alone, vehicle, anti-vimentin antibodies, alone, anti-nucleophosmin antibodies, alone, or anti-prohibitin antibodies, alone.

### Example 35

### Targeted Therapy Against SKOV3 Cells Using Vimentin, Prohibitin, Nucleophosmin, and Cytokeratin 8 Therapy in Combination with Taxol

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection ofthe cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of cell surface marker-directed treatment on tumor sensitivity to chemotherapeutic drugs. Mice are then treated with a combinatorial therapy in which antibodies directed against vimentin, prohibitin, nucleophosmin, and cytokeratin 8 are injected into mice having the SKOV3 tumors. Mice in the combinatorial therapy group are treated with 250 μCi each of: ¹³¹I-anti-vimentin antibodies, ¹³¹I-anti-prohibitin antibodies, ¹³¹I-anti-nucleophosmin antibodies, and ¹³¹I-anti-cytokeratin 8 antibodies, prepared as described above. Taxol is also administered to the combinatorial therapy group at concentrations of either 7.5 mg/kg or 10 mg/kg daily. Control mice receive vehicle, taxol, alone, (7.5 mg/kg or 10 mg/kg daily), 250 μCi of ¹³¹I-anti-vimentin antibodies, alone, 250 μCi of ¹³¹I-anti-nucleophosmin antibodies, alone, 250 μCi of ¹³¹I-anti-cytokeratin 8 antibodies, alone, or 250 μCi of ¹³¹I-anti-prohibitin antibodies, alone.

All treatments are introduced by intravenous administration. Mice are then sacrificed and tumors are excised for relative volume determinations. Relative tumor volume is determined as described above.

Tumors from mice treated with the combination of anti-vimentin, anti-nucleophosmin, anti-cytokeratin 8, and anti-prohibitin antibodies in combination with taxol have decreased volume when compared to tumors from mice treated with taxol, alone, vehicle, anti-vimentin antibodies, alone, anti-nucleophosmin antibodies, alone, anti-cytokertain 8, or anti-prohibitin antibodies, alone.

### Example 36

### Targeted Therapy Against SKOV3 Cells Using Vimentin, Prohibitin, Nucleophosmin, β-Tubulin, and Cytokeratin 8 Therapy in Combination with Taxol

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of cell surface marker-directed treatment on tumor sensitivity to chemotherapeutic drugs. Mice are then treated with a combinatorial therapy in which antibodies directed against vimentin, prohibitin, nucleophosmin, β-tubulin, and cytokeratin 8 are injected into mice having the SKOV3 tumors. Mice in the combinatorial therapy group are treated with 250 μCi each of: ¹³¹I-anti-vimentin antibodies, ¹³¹I-anti-prohibitin antibodies, ¹³¹I-anti-nucleophosmin antibodies, ¹³¹I-anti-β-tubulin antibodies, and ¹³¹I-anti-cytokeratin 8 antibodies, prepared as described above. Taxol is also administered to the combinatorial therapy group at concentrations of either 7.5 mg/kg or 10 mg/kg daily. Control mice receive vehicle, taxol, alone, (7.5 mg/kg or 10 mg/kg daily), 250 μCi of ¹³¹I-anti-vimentin antibodies, alone, 250 μCi of ¹³¹I-anti-nucleophosmin antibodies, alone, 250 μCi of ¹³¹I-anti-cytokeratin 8 antibodies, alone, 250 μCi of ¹³¹I-anti-β-tubulin antibodies, or 250 μCi of ¹³¹I-anti-prohibitin antibodies, alone.

All treatments are introduced by intravenous administration. Mice are then sacrificed and tumors are excised for relative volume determinations. Relative tumor volume is determined as described above.

Tumors from mice treated with the combination of anti-vimentin, anti-nucleophosmin, anti-cytokeratin 8, anti-β-tubulin, and anti-prohibitin antibodies in combination with taxol have decreased volume when compared to tumors from mice treated with taxol, alone, vehicle, anti-vimentin antibodies, alone, anti-nucleophosmin antibodies, alone, anti-cytokertain 8, anti-β-tubulin, or anti-prohibitin antibodies, alone.

### Example 37

### Targeted Therapy Against SKOV3 Cells Using Vimentin, Prohibitin, Nucleophosmin, β-Tubulin, Annexin I, and Cytokeratin 8 Therapy in Combination with Taxol

1. Treatment of SKOV3 Cancer Cells

In order to determine whether therapeutic agents directed against the cell-surface markers described above are useful in treating a preexisting cancerous condition, thymic, nude mice, 5 to 7 weeks old, receive an s.c. injection of the cells 5×10⁵ SKOV3 tumor cells, and tumors are allowed to form. Starting on the first day of treatment, tumor growth is measured by palpitation, and the volume of the xenograft is monitored every 4 days. Tumors are allowed to grow to a sufficient size (5.5 mm-100 mm) for appropriate analysis of the effects of cell surface marker-directed treatment on tumor sensitivity to chemotherapeutic drugs. Mice are then treated with a combinatorial therapy in which antibodies directed against vimentin, prohibitin, nucleophosmin, β-tubulin, and cytokeratin 8 are injected into mice having the SKOV3 tumors. Mice in the combinatorial therapy group are treated with 250 μCi each of: ¹³¹I-anti-vimentin antibodies, ¹³¹I-anti-prohibitin antibodies, ¹³¹I-anti-nucleophosmin antibodies, ¹³¹I-anti-β-tubulin antibodies, ¹³¹I-anti-annexin I antibodies, and ¹³¹I-anti-cytokeratin 8 antibodies, prepared as described above. Taxol is also administered to the combinatorial therapy group at concentrations of either 7.5 mg/kg or 10 mg/kg daily. Control mice receive vehicle, taxol, alone, (7.5 mg/kg or 10 mg/kg daily), 250 μCi of ¹³¹I-anti-vimentin antibodies, alone, 250 μCi of ¹³¹I-anti-β-tubulin antibodies, alone, 250 μCi of ¹³¹I-anti-cytokeratin 8 antibodies, alone, 250 μCi of ¹³¹I-anti-nucleophosmin antibodies, 250 μCi of ¹³¹I-anti-annexin I antibodies, or 250 μCi of ¹³¹I-anti-prohibitin antibodies, alone.

All treatments are introduced by intravenous administration. Mice are then sacrificed and tumors are excised for relative volume determinations. Relative tumor volume is determined as described above.

Tumors from mice treated with the combination of anti-vimentin, anti-nucleophosmin, anti-cytokeratin 8, anti-β-tubulin, anti-annexin I, and anti-prohibitin antibodies in combination with taxol have decreased volume when compared to tumors from mice treated with taxol, alone, vehicle, anti-vimentin antibodies, alone, anti-nucleophosmin antibodies, alone, anti-cytokertain 8, anti-β-tubulin, anti-annexin I, or anti-prohibitin antibodies, alone.

### Equivalents

Those skilled in the art will recognize, or be able to ascertain, using no more than routine experimentation, numerous equivalents to the specific compositions and procedures described herein. Such equivalents are considered to be within the scope of this invention, and are covered by the following claims.

1. A method of treating and/or preventing a neoplasm in a patient, comprising administering an effective amount of one or more therapeutic agents, each of which binds specifically to a protein target from the group consisting of nucleophosmin, HSC70, BIP, Grp75, PDI ER60 precursor, HSP60, TCP-1ε, ERp29, HSP27, vimentin, α-internexin, cytokeratin 8, β-actin, γ-actin, β-tubulin, nm23-H1, valosin containing protein, tumor protein D52-like 2, ASF-2, hnRNPK, hnRNPC, 24.1D5 antigen, hnRNPA/B, Eukaryotic Elongation Factor 1δ Isoform 2, AU-rich element RNA binding protein, Rad 23 homologue B, annexin I, prohibitin, ubiquilin 1, and thioredoxin peroxidase 4, wherein the protein target is on the surface of the neoplasm.

2. The method of claim 1, wherein the therapeutic agents are selected from the group consisting of ligands, nucleic acids, synthetic small molecules, peptidomimetic compounds, inhibitors, peptides, proteins, and antibodies or antigen-binding fragments thereof.

3. The method of claim 2, wherein two or more therapeutic agents are administered together simultaneously.

4. The method of claim 1, wherein the two or more therapeutic agents are bound to the neoplasm is internalized into the neoplastic cell.

5. The method of claim 1, wherein the two or more therapeutic agents are incorporated into a liposome.

6. The method of claim 5, wherein the liposome comprises a neoplastic cell-targeting agent on its surface.

7. The method of claim 6, wherein the neoplastic cell-targeting agent comprises an antibody, or antigen-binding fragment thereof, specific for at least one cell marker selected from the group consisting of multidrug resistance protein 1, BRCP, p53, vimentin, α-enolase, nucleophosmin, and HSC70.

8. The method of claim 1, wherein the therapeutic agent(s) is administered to the patient by injection at the site of the neoplasm.

9. The method of claim 1, wherein the therapeutic agent(s) is administered to the patient by surgical introduction at the site of the neoplasm.

10. The method of claim 1, wherein the therapeutic agent(s) is administered to the patient by inhalation of an aerosol or vapor.

11. The method of claim 1, wherein the neoplasm is selected from the group consisting of breast adenocarcinoma, breast carcinoma, ovarian carcinoma, ovarian adenocarcinoma, lung small cell carcinoma, lung carcinoma, and leukemia.

12. The method of claim 11, wherein the neoplasm comprises breast adenocarcinoma.

13. The method of claim 11, wherein the neoplasm comprises ovarian adenocarcinoma.

14. The method of claim 1, wherein two or more therapeutic agents directed are administered to a patient in need thereof.

15. The method of claim 1, wherein three or more therapeutic agents are administered to a patient in need thereof.

16. The method of claim 1, wherein four or more therapeutic agents are administered to a patient in need thereof.

17. The method of claim 1, wherein five or more therapeutic agents are administered to a patient in need thereof.

18. The method of claim 1, wherein six or more therapeutic agents are administered to a patient in need thereof.

19. The method of claim 1, wherein seven or more therapeutic agents are administered to a patient in need thereof.

20. The method of claim 1, wherein eight or more therapeutic agents are administered to a patient in need thereof.

21. The method of claim 1, wherein nine or more therapeutic agents are administered to a patient in need thereof.

22. The method of claim 1, wherein ten or more therapeutic agents are administered to a patient in need thereof.

23. The method of claim 1, wherein eleven or more therapeutic agents are administered to a patient in need thereof.

24. The method of claim 1, wherein twelve or more therapeutic agents are administered to a patient in need thereof.

25. The method of claim 1, wherein thirteen or more therapeutic agents are administered to a patient in need thereof.

26. The method of claim 1, wherein fourteen or more therapeutic agents are administered to a patient in need thereof.

27. The method of claim 1, wherein fifteen or more therapeutic agents are administered to a patient in need thereof.

28. The method of claim 1, wherein sixteen or more therapeutic agents are administered to a patient in need thereof.

29. The method of claim 1, wherein seventeen or more therapeutic agents are administered to a patient in need thereof.

30. The method of claim 1, wherein eighteen or more therapeutic agents are administered to a patient in need thereof.

31. The method of claim 1, wherein nineteen or more therapeutic agents are administered to a patient in need thereof.

32. The method of claim 1, wherein twenty or more therapeutic agents are administered to a patient in need thereof.

33. The method of claim 1, wherein twenty-one or more therapeutic agents are administered to a patient in need thereof.

34. The method of claim 1, wherein twenty-two or more therapeutic agents are administered to a patient in need thereof.

35. The method of claim 1, wherein twenty-three or more therapeutic agents are administered to a patient in need thereof.

36. The method of claim 1, wherein twenty-four or more therapeutic agents are administered to a patient in need thereof.

37. The method of claim 1, wherein twenty-five or more therapeutic agents are administered to a patient in need thereof.

38. The method of claim 1, wherein twenty-six or more therapeutic agents are administered to a patient in need thereof.

39. The method of claim 1, wherein twenty-seven or more therapeutic agents are administered to a patient in need thereof.

40. The method of claim 1, wherein twenty-eight or more therapeutic agents are administered to a patient in need thereof.

41. The method of claim 1, wherein all therapeutic agents are administered to a patient in need thereof.

42. The method of claim 1, wherein one or more therapeutic agents are administered simultaneously or sequentially with a chemotherapeutic drug.

43. The method of claim 42, wherein the chemotherapeutic drug is selected from the group consisting of Actinomycin, Adriamycin, Altretamine, Asparaginase, Bleomycin, Busulfan, Capecitabine, Carboplatin, Carmustine, Chlorambucil, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastine, Vincristine, Vinorelbine, and combinations thereof.

44. A pharmaceutical formulation for treating a neoplasm, comprising:
a) at least one therapeutic agent directed against a protein target protein target from the group consisting of nucleophosmin, HSC70, BIP, Grp75, PDI ER60 precursor, HSP60, TCP-1ε, ERp29, HSP27, vimentin, α-internexin, cytokeratin 8, β-actin, γ-actin, β-tubulin, nm23-H1, valosin containing protein, tumor protein D52-like 2, ASF-2, hnRNPK, hnRNPC, 24.1D5 antigen, hnRNPA/B, Eukaryotic Elongation Factor 1δ Isoform 2, AU-rich element RNA binding protein, Rad 23 homologue B, annexin I, prohibitin, ubiquilin 1, and thioredoxin peroxidase 4;
b) a chemotherapeutic drug; and
c) a pharmaceutically acceptable carrier.

45. The pharmaceutical formulation of claim 44, wherein the therapeutic agents are selected from the group consisting of ligands, nucleic acids, synthetic small molecules, peptidomimetic compounds, inhibitors, peptides, proteins, and antibodies or antigen-binding fragments thereof.

46. The pharmaceutical formulation of claim 44, wherein the therapeutic agents are incorporated into a liposome.

47. The pharmaceutical formulation of claim 46, wherein the liposome comprises a neoplastic cell-targeting component on its surface.

48. The pharmaceutical formulation of claim 47, wherein the neoplastic cell-targeting component is an antibody, or antigen-binding fragment thereof, that binds to a neoplastic cell marker selected from the group consisting of multidrug resistance protein 1, BRCP, p53, vimentin, α-enolase, nucleophosmin, and HSC70.

49. The pharmaceutical formulation of claim 44, wherein the chemotherapeutic drug is selected from the group consisting of Actinomycin, Adriamycin, Altretamine, Asparaginase, Bleomycin, Busulfan, Capecitabine, Carboplatin, Carmustine, Chlorambucil, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastine, Vincristine, Vinorelbine, and combinations thereof.
